SEQUENCE LISTING (15 sequences)

SEQ ID NO: 1 - protein, 515 aa, Brachydanio rerio
MVLDLDLFRTDKGGDPEIIRETQRKRFKDVSLVDKLVQADTEWRKCRFTADNLNKAKNLCSKSIGEKMKKKEPVGDDDTLPEEAQNLEALTAETLSPLTVTQIKKVRVLVDEAVQKTDSDRLKLEAERFEYLREIGNLLHPSVPISNDEDADNKVERTWGDCTVQKKYSHVDLVVMVDGYEGEKGAIVAGSRGYFLKGPLVFLEQALINYALRILYSKNYNLLYTPFFMRKEVMQEVAQLSQFDEELYKVIGKGSEKSDDNTVDEKYLIATSEQPIAAFLRDEWLKPEELPIRYAGLSTCFRQEVGSHGRDTRGIFRVHQFEKIEQFVYASPHDGKSWEMFDEMIGTAESFYQTLGIPYRIVNIVSGALNHAASKKLDLEAWFPGSQAFRELVSCSNCTDYQARRLRIRYGQTKKMMDKAEFVHMLNATMCATTRVICAILENFQTEEGIIVPEPLKAFMPPGLTEIIKFVKPAPIDQETTKKQKKQQEGGKKKKHQGGDADLENKVENMSVNDS

SEQ ID NO: 2 - DNA, 1926 bp, Brachydanio rerio
tccgcaccgcgcacctcgtccacaggcataatggtgctcgatttagacctgtttcgcaccgacaaaggcggcgatcctgaaattatccgggaaactcagaggaaacggttcaaagatgtgtctctggtggataaactggtccaggcggacacagaatggagaaaatgtcgtttcacagcagataaccttaacaaggccaagaatctctgcagcaaatccatcggtgaaaagatgaagaagaaagagccagtaggggatgatgacactcttccagaagaggctcagaatctggaagccctcactgcagaaacgttatcgccgcttactgtgactcagataaagaaagtgcgggttctggtggatgaggctgtgcagaagacagacagtgaccggctgaagctggaggcagagcgctttgagtatctgcgagagatcggcaacctcctacatccctctgtgcccatcagcaacgatgaggatgctgataataaagtggagcgcacctggggtgactgcacggtgcagaagaagtactctcatgtggacctggtcgtcatggttgatggatatgagggggaaaaaggagccattgttgctggaagcagaggatactttctcaaggggcctttagtgttcttggagcaagctttgattaactatgcgctgcggatcctgtacagcaagaactacaacctcctgtacacacccttcttcatgaggaaagaagtcatgcaggaggtcgctcagctcagccagtttgacgaggagctctacaaggtgatcgggaaaggaagtgagaagtctgatgataacacagtggacgagaagtacttgattgccacatcagagcagccaatcgcagccttcctgagagatgagtggctgaagccagaagaacttcctatccgctacgctggcctctccacctgcttcagacaggaagtgggctctcatggcagagacacgcgcgggatcttcagggtccatcagtttgagaagattgagcagtttgtgtacgcctctcctcatgatggcaaatcctgggagatgtttgatgaaatgattggaaccgctgaatccttttatcaaacactaggaattccttatcgaattgtcaacatcgtgtcaggtgctttgaaccacgcagctagtaaaaagctggatttagaggcttggtttcctggttcccaggcttttagagagcttgtgtcatgctcaaactgtacagactatcaggctcgtcgcttgcggattcgatacgggcagactaagaaaatgatggacaaggctgagtttgtgcacatgctcaatgccaccatgtgtgcgaccactcgtgtcatctgtgccatcctggagaacttccaaacagaggaaggcatcattgttccagaacccctcaaggcattcatgcctccaggtttaacagaaataatcaagtttgtgaagccagcccccattgaccaggaaacaacaaagaagcagaagaaacagcaggaaggaggaaagaagaagaaacatcagggcggcgatgctgatctagagaacaaagtggagaacatgtctgtcaatgactcttagacacgccctccatagtctcatccaatcatattggttcacaggttcttcatttcgtgtaacccgatcacaattgctgtcccctggagctctcactttttcatccaggacagtcctactggaactaaaggtgatgctgtcatgcttataatcttatctcacatcaaccaatcattttcatgccaaggggtctttagaaatattcaattaaatgcatggtgacaagacatttagccattagacggaaatgcttttacagcactttaatttcctgaaggcactgcatttcaaacctgccaatgaattaaagggaacatgacagtcagtacctacccgggcggccgctcgagg

SEQ ID NO: 3 - DNA, 21 bp, Artificial Sequence
  Primer sequence
cgctggaaaggaccttacaca

SEQ ID NO: 4 - DNA, 20 bp, Artificial Sequence
  Primer sequence
tgcgatgccgtctactttga

SEQ ID NO: 5 - DNA, 20 bp, Artificial Sequence
  Primer sequence
ctgctgaccacccccaccgc

SEQ ID NO: 6 - DNA, 26 bp, Artificial Sequence
  Primer sequence
attggagaagtctaccagaagcctaa

SEQ ID NO: 7 - DNA, 23 bp, Artificial Sequence
  Primer sequence
cttagttgcttgtccagggttga

SEQ ID NO: 8 - DNA, 17 bp, Artificial Sequence
  Primer sequence
gcgcaacggcggcgctc

SEQ ID NO: 9 - DNA, 30 bp, Artificial Sequence
  Primer sequence
ccatgccttgcaaaatggcgttacttaagc

SEQ ID NO: 10 - DNA, 21 bp, Artificial Sequence
  Primer sequence
ccgcaaccctgggagacgtcc

SEQ ID NO: 11 - DNA, 30 bp, Artificial Sequence
  Primer sequence
tgatctcgagccaaacctacaggtggggtc

SEQ ID NO: 12 - DNA, 20 bp, Artificial Sequence
  Primer sequence
gtggttctggtaggagacga

SEQ ID NO: 13 - DNA, 23 bp, Artificial Sequence
  Primer sequence
cgtgacatgacaccgtaagtgtg

SEQ ID NO: 14 - DNA, 21 bp, Artificial Sequence
  Primer sequence
gtccaggcggacacagaatgg

SEQ ID NO: 15 - DNA, 30 bp, Artificial Sequence
  Primer sequence
tgatctcgagttccttgggagggtctcctc

(19) United States
(12) Patent Application Publication
(10) Pub. No.: US 2004/0142440 A1
(43) Pub. Date: Jul. 22, 2004

(54) Seryl transfer RNA synthetase polynucleotides and polypeptides and methods of use thereof

(75) Inventors: Nancy H. Hopkins, Cambridge, MA (US); Adam H. Amsterdam, Cambridge, MA (US); Eric C. Swindell, Sugar Land, TX (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Massachusetts Institute of Technology, Cambridge, MA

(21) Appl. No.: 10/635,145

(22) Filed: Aug. 6, 2003

Related U.S. Application Data
(60) Provisional application No. 60/401,556, filed on Aug. 6, 2002.

(51) Int. Cl. C12Q 1/68; C07H 21/04; C12N 9/10

(52) U.S. Cl. 435/193; 435/69.1; 435/320.1; 435/325; 536/23.2; 435/6

(57) ABSTRACT

Novel seryl transfer RNA polynucleotides and polypeptides, as well as methods of using such polynucleotides and polypeptides for the identification of therapeutic compounds are disclosed. Also disclosed are zebrafish that have a mutation in a seryl transfer RNA gene.

### RELATED APPLICATION

This application claims the benefit of U.S. Provisional Application No. 60/401,556, filed Aug. 6, 2002. The entire teachings of the above application are incorporated herein by reference.

### GOVERNMENT SUPPORT

The invention was supported, in whole or in part, by a grant R01-RR12589-05 from the National Institutes of Health. The Government has certain rights in the invention.

### BACKGROUND OF THE INVENTION

Genetic screens have been the most successful approach for identifying genes required for developmental processes. Applied on a sufficiently large scale, a genetic screen can identify all of the genes which, when mutated one at a time, impact the phenotype of interest. Genetic screens make no assumptions about the genes involved in the biological processes of interest and thus can reveal novel genetic pathways underlying important phenotypes.

Although it has long been the primary method for identifying the genetic basis of phenotypes in invertebrate organisms, genetic screening is rarely performed in vertebrate animals, and a saturation screen has never been achieved in any vertebrate. This is because the number of animals that must be raised, maintained, and screened is hundreds of thousands for a moderate-sized screen, and millions to achieve saturation. Nonetheless, many small-scale screens in zebrafish and mice have been highly successful, and two large-scale screens have been carried out in the zebrafish. The genetic screens that have already been performed in vertebrate animals hint at the great potential of this approach.

In zebrafish, simple visual screens of embryos in the first 5 days after fertilization can reveal mutations in genes essential for the normal development of most of the major organ systems, including the nervous system, heart, blood, gut, liver, jaws, eyes, and ears. Simply identifying mutant phenotypes in a genetic screen can be informative by revealing both the kinds of phenotypes that can occur and the number of genes involved in the process of interest. However, to understand how genes specify a biological process, it is essential to identify the mutated genes.

Insertional mutagenesis screens greatly speed the cloning of mutant genes. The integration of exogenous DNA sequences into a genome can be mutagenic, and the inserted DNA serves as a tag to clone mutated genes. Thus, genes involved in the development of zebrafish can be readily identified.

### SUMMARY OF THE INVENTION

To identify genes involved in the development of zebrafish, a large-scale insertional mutagenesis screen has been performed as described herein. One of the genes identified through this screen is a seryl transfer RNA (tRNA) synthetase gene. The mutation of this gene in zebrafish results in a phenotype in which blood circulates through the heart and part of the head, but does not circulate through the trunk of the zebrafish. Rather, the blood exits the heart, travels through a short loop in the area of the brachial arches and re-enters the heart.

Accordingly, the present invention relates to isolated or recombinant seryl tRNA synthetase polypeptides, and isolated seryl tRNA synthetase nucleic acid molecules encoding those polypeptides, as well as to vectors and cells containing those isolated nucleic acid molecules. The invention also relates to methods of modulating expression of seryl tRNA synthetase nucleic acid molecules and screens for identifying modulators of seryl tRNA synthetase expression.

Thus, in one aspect, the invention features an isolated seryl tRNA synthetase polypeptide or a biologically active fragment thereof. In one embodiment, the isolated seryl tRNA synthetase polypeptide has at least 82% amino acid identity to the amino acid sequence of SEQ ID NO: 1. In another embodiment, the polypeptide comprises or consists of the sequence of SEQ ID NO: 1. In still another embodiment, the polypeptide is a zebrafish polypeptide.

In another aspect, the invention features an isolated seryl tRNA synthetase polypeptide comprising the sequence of SEQ ID NO: 1.

In another aspect, the invention features an isolated seryl tRNA synthetase polypeptide consisting of the sequence of SEQ ID NO: 1.

In another aspect, the invention features an isolated polypeptide encoded by the DNA sequence of SEQ ID NO: 2.

In another aspect, the invention features an isolated nucleic acid molecule encoding a seryl tRNA synthetase polypeptide or a biologically active fragment thereof. In one embodiment, the encoded seryl tRNA synthetase polypeptide has at least 82% amino acid identity to the amino acid sequence of SEQ ID NO: 1. In another embodiment, the isolated nucleic acid comprises or consists of the sequence of SEQ ID NO: 2. In still another embodiment, the nucleic acid molecule is zebrafish nucleic acid molecule.

In another aspect, the invention features an isolated nucleic molecule encoding the polypeptide sequence of SEQ ID NO: 1 or a biologically active fragment thereof.

In still another embodiment, the invention features an isolated nucleic acid molecule selected from the group consisting of: a complement of an isolated nucleic acid molecule encoding a seryl tRNA synthetase polypeptide; the complement of an isolated nucleic acid molecule comprising the sequence of SEQ ID NO: 2; the complement of an isolated nucleic acid consisting of a nucleic acid of SEQ ID NO: 2; the complement of a nucleic acid molecule encoding the polypeptide of SEQ ID NO: 1; a nucleic acid sequence that is hybridizable under high stringency conditions to a nucleic acid molecule encoding a seryl tRNA synthetase polypeptide; a nucleic acid sequence that is hybridizable under high stringency conditions to a nucleic acid molecule comprising the sequence of SEQ ID NO: 2; a nucleic acid sequence that is hybridizable under high stringency conditions to a nucleic acid molecule consisting of the sequence of SEQ ID NO: 2; and a nucleic acid sequence that is hybridizable under high stringency conditions to a nucleic acid molecule encoding the polypeptide sequence of SEQ ID NO: 1. In one embodiment, the encoded seryl tRNA synthetase has at least 82% amino acid identity to the amino acid sequence of SEQ ID NO: 1. In another embodiment, the nucleic acid molecule is a zebrafish nucleic acid molecule.

In another aspect, the invention features a vector comprising any one of the nucleic acid molecules described above, as well as a cell containing such a vector.

In another aspect, the invention features a mutated seryl tRNA synthetase gene, wherein the mutation results in decreased seryl tRNA synthetase biological activity and/or levels. In one embodiment, the mutation is in an intron of the seryl tRNA synthetase gene, for example, between the first and second introns. In another embodiment, the mutation is a proviral insertion in an intron of the gene.

In still another aspect, the invention features a zebrafish comprising a mutated seryl tRNA synthetase gene. In one embodiment, the mutation results in decreased seryl tRNA synthetase polypeptide biological activity and/or levels. In another embodiment, the mutation is in an intron of a seryl tRNA synthetase gene, for example, between the first and second introns. The mutation can be, for example, a proviral insertion in an intron. In another embodiment, the zebrafish comprises a mutation resulting in a phenotype in which blood circulates through the heart of the zebrafish and through a short loop in the area of the branchial arches and re-enters the heart, without circulating throughout the trunk of the zebrafish. In one embodiment, the phenotype results from altered vasculature. In another embodiment, the zebrafish has altered angiogenic activity.

In yet another aspect, the invention features an antibody that selectively binds a serve tRNA synthetase polypeptide. In one embodiment, the polypeptide has at least 82% amino acid identity to SEQ ID NO: 1.

In still another embodiment, the invention features a method of identifying a compound that modulates expression of a seryl tRNA synthetase nucleic acid molecule, comprising contacting the nucleic acid molecule, or a cell or animal containing the nucleic acid molecule, with a candidate compound under conditions suitable for expression of the nucleic acid molecule; and assessing the level of expression of the nucleic acid molecule. A candidate compound that increases or decreases expression of the seryl tRNA synthetase nucleic acid molecule relative to a control is a compound that modulates expression of the seryl tRNA synthetase nucleic acid molecule.

In another aspect, the invention features a method of identifying a compound that modulates the seryl tRNA synthetase biological activity, for example, the enzymatic activity of a seryl tRNA synthetase polypeptide, comprising contacting the polypeptide or a biologically active fragment thereof, or a cell or animal containing the polypeptide or a biologically active fragment, with a candidate compound under conditions suitable for seryl tRNA synthetase biological activity, for example, enzymatic activity; and assessing the seryl tRNA synthetase biological activity of the polypeptide or fragment. A candidate compound that increases or decreases the seryl tRNA synthetase biological activity level of the polypeptide or biologically active fragment thereof relative to a control is a compound that modulates the seryl tRNA synthetase biological activity of the polypeptide.

In another aspect, the invention features a method of identifying a compound that modulates the angiogenic activity of a seryl tRNA synthetase polypeptide, comprising contacting the polypeptide or a biologically active fragment thereof, or a cell or animal containing the polypeptide or a biologically active fragment, with a candidate compound under conditions suitable for angiogenic activity; and assessing the angiogenic activity of the polypeptide or fragment. A candidate compound that increases or decreases the angiogenic activity level of the polypeptide or biologically active fragment thereof relative to a control is a compound that modulates the angiogenic activity of the polypeptide.

In still another aspect, the invention features a method of identifying a compound that modulates expression of a nucleic acid molecule encoding a seryl tRNA synthetase polypeptide, comprising contacting a nucleic acid molecule comprising a promoter region of a nucleic acid molecule encoding a seryl tRNA synthetase polypeptide or functional part of a promoter region of a nucleic acid molecule encoding a seryl tRNA synthetase polypeptide operably linked to a reporter gene with a candidate compound; and assessing the level of the reporter gene. The nucleic acid molecule can be in a cell-free system or in a cell or animal. A candidate compound that increases or decreases expression of the reporter gene relative to a control is a compound that modulates expression of the nucleic acid molecule encoding a seryl tRNA synthetase polypeptide.

In another aspect, the invention features a method of identifying a polypeptide that interacts with a seryl tRNA synthetase polypeptide in a yeast two-hybrid system, comprising providing a first nucleic acid vector comprising a nucleic acid molecule encoding a DNA binding domain and a seryl tRNA synthetase polypeptide; providing a second nucleic acid vector comprising a nucleic acid encoding a transcription activation domain and a nucleic acid encoding a test polypeptide; contacting the first nucleic acid vector with the second nucleic acid vector in a yeast two-hybrid system; and assessing transcriptional activation in the yeast two-hybrid system. An increase in transcriptional activation relative to a control indicates that the test polypeptide is a polypeptide that interacts with a seryl tRNA synthetase polypeptide.

In any of the above screening methods, the method can be carried out in a cell or animal, for example, a zebrafish. Alternatively the method can be carried out in a cell-free system. In another embodiment, the polypeptides used in the methods have at least 82% amino acid identity to the amino acid sequence of SEQ ID NO: 1. In another embodiment, the nucleic acid molecule used in the method encodes a polypeptide having at least 82% amino acid identity to the amino acid sequence of SEQ ID NO: 1. In still another embodiment, a human seryl tRNA synthetase nucleic acid molecule or polypeptide is used.

Compounds and/or polypeptides identified in the above-described screening methods are also part of the present invention.

The invention also features a pharmaceutical composition comprising a seryl tRNA synthetase polypeptide described above.

In addition, the present invention features a method of diagnosing an angiogenic disease, a vascular disease, a heart disease, or a circulatory disease in a subject. The method comprises assessing the level of activity or expression of the seryl tRNA synthetase polypeptide described above or the level of the nucleic acid molecule described above in a sample obtained from an individual. If the level is altered relative to a control, then the subject has an altered likelihood of having an angiogenic disease, a vascular disease, a heart disease, or a circulatory disease relative to an individual who does not have an altered expression of the seryl tRNA synthetase gene. In one embodiment, the polypeptide level is assayed using immunohistochemistry techniques. In another embodiment, the nucleic acid molecule level is assayed using in situ hybridization techniques.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows the amino acid sequence of a zebrafish seryl tRNA synthetase polypeptide (SEQ ID NO: 1).

FIG. 2 shows the cDNA sequence of a zebrafish seryl tRNA synthetase nucleic acid molecule (SEQ ID NO: 2). The ATG start site is located at nucleotide 31. The mutation in the zebrafish mutant described herein is in an intron following nucleotide 168 of this cDNA sequence. This intron occurs between the first and second exons.

FIG. 3 is a scanned image of a zebrafish that does not contain a mutation in the seryl tRNA synthetase gene (wild-type; top) compared to a zebrafish that does contain a mutation in the seryl tRNA synthetase gene as described herein.

FIG. 4 is a scanned image of an agarose gel through which reverse transcriptase polymerase chain reaction (RT-PCR) products for seryl tRNA synthetase (SertRS) and actin have been electrophoresed. Lane 1 shows seryl tRNA synthetase and actin RT-PCR products, diluted 1:1000, from wild-type zebrafish; lane 2 shows seryl tRNA synthetase and actin RT-PCR products, diluted 1:100, from wild-type zebrafish; lane 3 shows seryl tRNA synthetase and actin RT-PCR products, diluted 1:10, from wild-type zebrafish; and lane 4 shows undiluted seryl tRNA synthetase and actin RT-PCR products from wild-type zebrafish. Lane 5 shows seryl tRNA synthetase and actin RT-PCR products, diluted 1:1000, from zebrafish having a mutated seryl tRNA synthetase gene; lane 6 shows seryl tRNA synthetase and actin RT-PCR products, diluted 1:100, from zebrafish having a mutated seryl tRNA synthetase gene; lane 7 shows seryl tRNA synthetase and actin RT-PCR products, diluted 1:10, from zebrafish having a mutated seryl tRNA synthetase gene; and lane 8 shows undiluted seryl tRNA synthetase and actin RT-PCR products from zebrafish having a mutated seryl tRNA synthetase gene. Lane 9 shows a molecular weight ladder. (VA=ventral aorta; AA=aortic arch vessels; PHS=primary head sinus; CCV=common cardinal vein)

FIG. 5A is a schematic representation of blood circulation in wild-type zebrafish at 3 days post fertilization. (VA=ventral aorta; AA=aortic arch vessels; PHS=primary head sinus; CCV=common cardinal vein)

FIG. 5B is a schematic representation of blood circulation in zebrafish having a mutated seryl tRNA synthetase gene at 3 days post fertilization. (VA=ventral aorta; AA=aortic arch vessels; PHS=primary head sinus; CCV=common cardinal vein)

FIG. 5C is a schematic representation of blood circulation in zebrafish having a mutated seryl tRNA synthetase gene at 3.5 days post fertilization. (VA=ventral aorta; AA=aortic arch vessels; PHS=primary head sinus; CCV=common cardinal vein)

FIG. 5D is a schematic representation of blood circulation in zebrafish having a mutated seryl tRNA synthetase gene at 4 days post fertilization. (VA=ventral aorta; AA=aortic arch vessels; PHS=primary head sinus; CCV=common cardinal vein)

### DETAILED DESCRIPTION OF THE INVENTION

As described herein, a large-scale insertional mutagenesis screen was performed on zebrafish. The insertional mutagenesis method involved infecting zebrafish embryos with a retrovirus, and breeding the fish such that the mutation caused by the retrovirus is brought to homozygosity. The fish were then visually inspected for mutations in genes essential for the normal development of major organ systems, including the nervous system, heart, blood, gut, liver, jaws, eyes, and ears. Once a mutant phenotype was observed, the inserted retroviral DNA was used as a tag to clone the mutated gene involved in the mutation.

One zebrafish mutant identified during this screen has a phenotype in which blood, which normally circulates through the heart, head, and trunk the zebrafish body, circulates through the heart and a portion of the head, bypassing the remainder of the zebrafish body. The mutated gene in this zebrafish mutant is a seryl tRNA synthetase gene. The cloning and characterization of this novel seryl tRNA synthetase is described herein.

Polypeptides of the Invention

The present invention features isolated or recombinant seryl tRNA synthetase polypeptides, and fragments, derivatives, and variants thereof, as well as polypeptides encoded by nucleotide sequences described herein (e.g., other variants). As used herein, the term “polypeptide” refers to a polymer of amino acids, and not to a specific length; thus, peptides, oligopeptides, and proteins are included within the definition of a polypeptide.

As used herein, a polypeptide is said to be “isolated,” “substantially pure,” or “substantially pure and isolated” when it is substantially free of cellular material, when it is isolated from recombinant or non-recombinant cells, or free of chemical precursors or other chemicals when it is chemically synthesized. In addition, a polypeptide can be joined to another polypeptide with which it is not normally associated in a cell (e.g., in a “fusion protein”) and still be “isolated,” “substantially pure,” or “substantially pure and isolated.” An isolated, substantially pure or substantially pure and isolated polypeptide may be obtained, for example, using affinity purification techniques described herein, as well as other techniques described herein and known to those skilled in the art.

By a “seryl tRNA synthetase polypeptide” is meant a polypeptide having seryl tRNA synthetase biological activity, for example, seryl tRNA synthetase enzymatic activity and/or angiogenesis modulating activity. A seryl tRNA synthetase polypeptide is also a polypeptide whose activity can be inhibited by molecules having seryl tRNA synthetase inhibitory activity. Examples of seryl tRNA synthetase polypeptides include a substantially pure polypeptide comprising or consisting of SEQ ID NO: 1; and a polypeptide having preferably at least 82%, 85%, 90%, 95%, or 99% sequence identity to SEQ ID NO: 1, as determined using the BLAST program and parameters described herein. A seryl tRNA synthetase gene is also a gene that comprises or consists of one or more domains that catalyzes aminoacylation of tRNAs. A seryl tRNA synthetase polypeptide is also a polypeptide encoded by the nucleic acid sequence of SEQ ID NO: 2. Other examples of tRNA synthetase polypeptides include those identified as GenBank Accession Numbers P49591 (human), XP 131123 (mouse), Q9GMB8 (cow), AAF51155 (drosophila), and NP 501804 (Caenorhabditis elegans).

In one embodiment, the seryl tRNA synthetase polypeptide has seryl tRNA synthetase enzymatic activity and/or angiogenesis modulatory activity. In one embodiment the seryl tRNA synthetase has one of the above biological activities. In another embodiment, the seryl tRNA synthetase has both of the above biological activities. As used herein, by “seryl tRNA synthetase enzymatic activity” is meant catalysis of aminoacylation of tRNAs. Methods for assessing seryl tRNA synthetase enzymatic activity are described, for example, by Sampson and Saks, Nucleic Acids Res. 21(19):4467-75 (1993); and Stefanska et al., J. Antibiot. (Tokyo) 53(12):1346-53 (2000), the entire teachings of which are incorporated by reference herein. As used herein, by “angiogenic modulatory activity” is meant increasing or decreasing angiogenesis (blood vessel formation) in a tissue (in vivo) or under in vitro conditions. In vitro and in vivo methods for detecting and/or measuring angiogenic activity are described, for example by Tanaka et al, Exp. Pathol. 30(3):143-50 (1986); Stallmach et al, Angiogenesis 4(1):79-84 (2001); McCarty et al., Int. J. Oncol. 21(1):5-10 (2002); Blacher et al., Angiogenesis 4(2):133-42 (2001); and Brown et al., Lab Invest. 75(4):539-55 (1996), the entire teachings of which are hereby incorporated by reference herein.

A polypeptide of the invention can be purified to homogeneity. It is understood, however, that preparations in which the polypeptide is not purified to homogeneity are useful. The critical feature is that the preparation allows for the desired function of the polypeptide, even in the presence of considerable amounts of other components. Thus, the invention encompasses various degrees of purity. In one embodiment, the language “substantially free of cellular material” includes preparations of the polypeptide having less than about 30% (by dry weight) other proteins (i.e., contaminating protein), less than about 20% other proteins, less than about 10% other proteins, or less than about 5% other proteins.

When a polypeptide is recombinantly produced, it can also be substantially free of culture medium, i.e., culture medium represents less than about 20%, less than about 10%, or less than about 5% of the volume of the polypeptide preparation. The language “substantially free of chemical precursors or other chemicals” includes preparations of the polypeptide in which it is separated from chemical precursors or other chemicals that are involved in its synthesis. In one embodiment, the language “substantially free of chemical precursors or other chemicals” includes preparations of the polypeptide having less than about 30% (by dry weight) chemical precursors or other chemicals, less than about 20% chemical precursors or other chemicals, less than about 10% chemical precursors or other chemicals, or less than about 5% chemical precursors or other chemicals.

In one embodiment, a polypeptide of the invention comprises an amino acid sequence encoded by a nucleic acid molecule of SEQ ID NO: 2, and complements and portions thereof. The polypeptides of the invention also encompasses fragments and sequence variants. Variants include a substantially homologous polypeptide encoded by the same genetic locus in an organism, i.e., an allelic variant, as well as other variants. Variants also encompass polypeptides derived from other genetic loci in an organism, but having substantial homology to a polypeptide encoded by a nucleic acid molecule comprising the nucleotide sequence of SEQ ID NO: 2, and complements and portions thereof, or having substantial homology to a polypeptide encoded by a nucleic acid molecule comprising the nucleotide sequence of SEQ ID NO: 2. Variants also include polypeptides substantially homologous or identical to these polypeptides but derived from another organism, i.e., an ortholog. Variants also include polypeptides that are substantially homologous or identical to these polypeptides that are produced by chemical synthesis. Variants also include polypeptides that are substantially homologous or identical to these polypeptides that are produced by recombinant methods.

As used herein, two polypeptides (or a region of the polypeptides) are substantially homologous or identical when the amino acid sequences are at least about 82%, 85%, 90%, 95%, or 99% homologous or identical. A substantially identical or homologous amino acid sequence, according to the present invention, will be encoded by a nucleic acid molecule hybridizing to SEQ ID NO: 2, or a portion thereof, under stringent conditions as more particularly described herein.

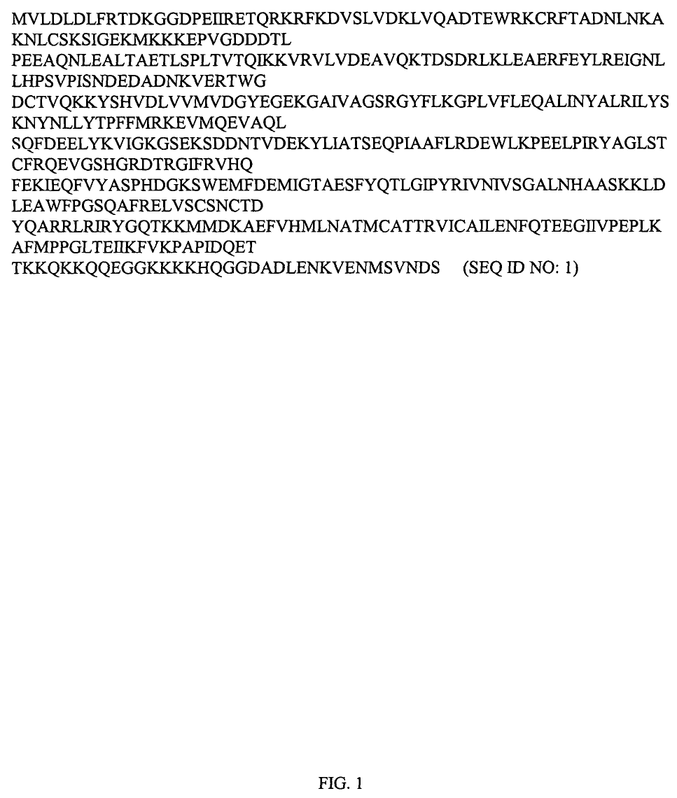

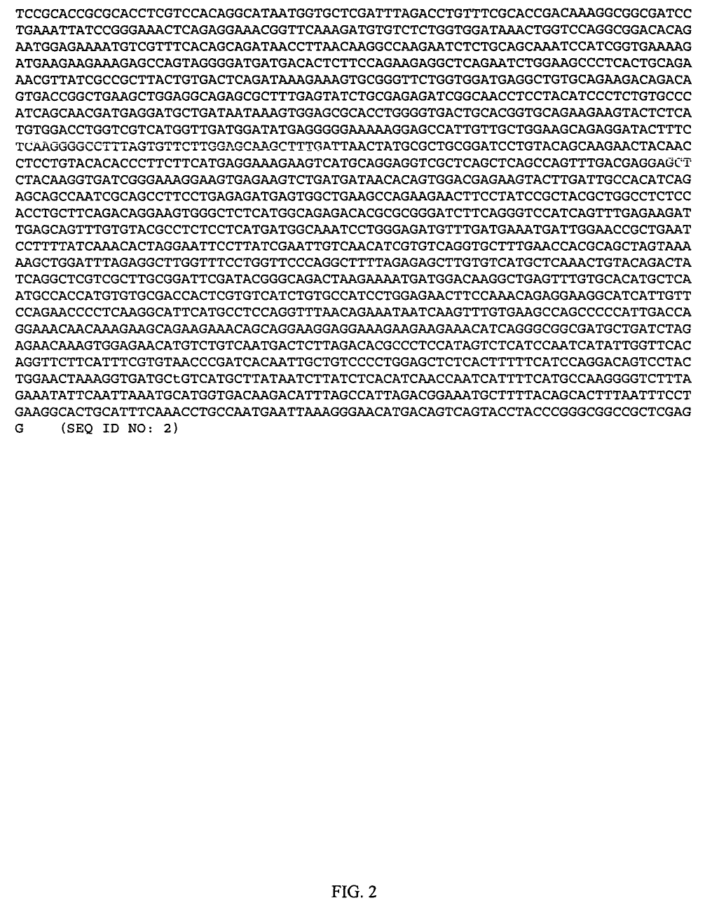

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of the seryl tRNA synthetase amino acid or nucleotide sequence aligned for comparison purposes is at least 30%, 40%, 50%, 60%,70%, 80%, 90%, 95%, or 100% of the length of the reference sequence, for example, those sequences provided in FIGS. 1 and 2. The actual comparison of the two sequences can be accomplished by well-known methods, for example, using a mathematical algorithm. A preferred, non-limiting example of such a mathematical algorithm is described in Karlin et al., Proc. Natl. Acad. Sci. USA, 90:5873-5877 (1993). Such an algorithm is incorporated into the BLASTN and BLASTX programs (version 2.2) as described in Schaffer et al., Nucleic Acids Res., 29:2994-3005 (2001). When utilizing BLAST and Gapped BLAST programs, the default parameters of the respective programs (e.g., BLASTN) can be used. In one embodiment, the database searched is a non-redundant (NR) database, and parameters for sequence comparison can be set at: no filters; Expect value of 10; Word Size of 3; the Matrix is BLOSUM62; and Gap Costs have an Existence of 11 and an Extension of 1. In another embodiment, the percent identity between two polypeptides or two polynucleotides is determined over the full-length of the polypeptide or polynucleotide of interest.

Another preferred, non-limiting example of a mathematical algorithm utilized for the comparison of sequences is the algorithm of Myers and Miller, CABIOS (1989). Such an algorithm is incorporated into the ALIGN program (version 2.0), which is part of the GCG sequence alignment software package (Accelrys, San Diego, Calif.). When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, Comput. Appl. Biosci., 10: 3-5 (1994); and FASTA described in Pearson and Lipman, Proc. Natl. Acad. Sci USA, 85: 2444-8 (1988).

In another embodiment, the percent identity between two amino acid sequences can be accomplished using the GAP program in the GCG software package using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package, using a gap weight of 50 and a length weight of 3.

The invention also encompasses seryl tRNA synthetase polypeptides having a lower degree of identity but having sufficient similarity so as to perform one or more of the same functions performed by a seryl tRNA synthetase polypeptide encoded by a nucleic acid molecule of the invention. Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu, and Ile; interchange of the hydroxyl residues Ser and Thr; exchange of the acidic residues Asp and Glu; substitution between the amide residues Asn and Gln; exchange of the basic residues Lys and Arg; and replacements among the aromatic residues Phe and Tyr. Guidance concerning which amino acid changes are likely to be phenotypically silent are found in Bowie et al., Science 247: 1306-1310 (1990).

A variant polypeptide can differ in amino acid sequence by one or more substitutions, deletions, insertions, inversions, fusions, and truncations or a combination of any of these. Further, variant polypeptides can be fully functional or can lack function in one or more activities, for example, in seryl tRNA synthetase enzymatic activity or angiogenesis modulatory activity. Fully functional variants typically contain only conservative variation or variation in non-critical residues or in non-critical regions. Functional variants can also contain substitution of similar amino acids that result in no change or an insignificant change in function. Alternatively, such substitutions may positively or negatively affect function to some degree. Non-functional variants typically contain one or more non-conservative amino acid substitutions, deletions, insertions, inversions, or truncations or a substitution, insertion, inversion, or deletion in a critical residue or critical region, such critical regions include the domains that provides the polypeptide with aminoacylation of tRNA catalysis activity. Such domains have been described in the art, and generally include a nucleotide binding fold which is the active site for interaction with the acceptor stem of tRNA, and a domain which associates with the anticodon arm of the tRNA molecule.

Amino acids that are essential for function can be identified by methods known in the art, such as site-directed mutagenesis or alanine-scanning mutagenesis (Cunningham et al., Science, 244: 1081-1085 (1989)). The latter procedure introduces a single alanine mutation at each of the residues in the molecule (one mutation per molecule). The resulting mutant molecules are then tested for biological activity in vitro. Sites that are critical for polypeptide activity can also be determined by structural analysis, such as crystallization, nuclear magnetic resonance, or photoaffinity labeling (See Smith et al., J. Mol. Biol., 224: 899-904 (1992); and de Vos et al. Science, 255: 306-312 (1992)).

The invention also includes seryl tRNA synthetase polypeptide fragments of the polypeptides of the invention. Fragments can be derived from a polypeptide comprising SEQ ID NO: 1, or from a polypeptide encoded by a nucleic acid molecule comprising SEQ ID NO: 2, or a portion thereof, complements thereof, or other variant thereof. The present invention also encompasses fragments of the variants of the polypeptides described herein. Useful fragments include those that retain one or more of the biological activities of the polypeptide, as well as fragments that can be used as an immunogen to generate polypeptide-specific antibodies.

Biologically active fragments (peptides that are, for example, 6, 9, 12, 15, 18, 20, 30, 40, 50, 60, 70, 80, 90, 100, or more amino acids in length) can comprise a domain, segment, or motif, for example, a seryl tRNA synthetase domain, that has been identified by analysis of the polypeptide sequence using well-known methods.

Fragments can be discrete (not fused to other amino acids or polypeptides) or can be fused to one or more components of a polypeptide. Further, several fragments can be comprised within a single larger polypeptide. In one embodiment, a fragment designed for expression in a host can have heterologous pre- and pro-polypeptide regions fused to the amino terminus of the polypeptide fragment and an additional region fused to the carboxyl terminus of the fragment.

Standard molecular biology methods for generating polypeptide fragments are known in the art. Once the fragments are generated, they can be tested for biological activity, using, for example, any of the methods described herein.

The invention thus provides chimeric or fusion polypeptides. These comprise a seryl tRNA synthetase polypeptide of the invention operatively linked to a heterologous protein or polypeptide having an amino acid sequence not substantially homologous to the polypeptide. “Operatively linked” indicates that the polypeptide and the heterologous protein are fused in-frame. The heterologous protein can be fused to the N-terminus or C-terminus of the polypeptide. In one embodiment, the fusion polypeptide does not affect the function of the polypeptide per se. For example, the fusion polyneptide can be a GST-fusion polypeptide in which the polypeptide sequences are fused to the C-terminus of the GST sequences. Other types of fusion polypeptides include, but are not limited to, enzymatic fusion polypeptides, for example, β-galactosidase fusions, yeast two-hybrid GAL fusions, poly-His fusions, FLAG-tagged fusions and Ig fusions. Such fusion polypeptides can facilitate the purification of recombinant polypeptide. In certain host cells (e.g., mammalian host cells), expression and/or secretion of a polypeptide can be increased by using a heterologous signal sequence. Therefore, in another embodiment, the fusion polypeptide contains a heterologous signal sequence at its N-terminus.

EP-A 0464 533 discloses fusion proteins comprising various portions of immunoglobulin constant regions. The Fc is useful in therapy and diagnosis and thus results, for example, in improved pharmacokinetic properties (EP-A 0232 262). In drug discovery, for example, human proteins have been fused with Fc portions for the purpose of high-throughput screening assays to identify antagonists. (See Bennett et al., Journal of Molecular Recognition, 8: 52-58 (1995) and Johanson et al., The Journal of Biological Chemistry, 270,16: 9459-9471 (1995)). Thus, this invention also encompasses soluble fusion polypeptides containing a polypeptide of the invention and various portions of the constant regions of heavy or light chains of immunoglobulins of various subclass (IgG, IgM, IgA, IgE).

A chimeric or fusion polypeptide can be produced by standard recombinant DNA techniques. For example, DNA fragments coding for the different polypeptide sequences are ligated together in-frame in accordance with conventional techniques. In another embodiment, the fusion gene can be synthesized by conventional techniques including automated DNA synthesizers. Alternatively, PCR amplification of nucleic acid fragments can be carried out using anchor primers that give rise to complementary overhangs between two consecutive nucleic acid fragments that can subsequently be annealed and re-amplified to generate a chimeric nucleic acid sequence (see Ausubel et al., “Current Protocols in Molecular Biology,” John Wiley & Sons, (1998), the entire teachings of which are incorporated by reference herein). Moreover, many expression vectors are commercially available that already encode a fusion moiety (e.g., a GST protein). A nucleic acid molecule encoding a polypeptide of the invention can be cloned into such an expression vector such that the fusion moiety is linked in-frame to the polypeptide.

The substantially pure, isolated, or substantially pure and isolated seryl tRNA synthetase polypeptide can be purified from cells that naturally express it, purified from cells that have been altered to express it (recombinant), or synthesized using known protein synthesis methods. In one embodiment, the polypeptide is produced by recombinant DNA techniques. For example, a nucleic acid molecule encoding the polypeptide is cloned into an expression vector, the expression vector is introduced into a host cell, and the polypeptide is expressed in the host cell. The polypeptide can then be isolated from the cells by an appropriate purification scheme using standard protein purification techniques.

In general, seryl tRNA synthetase polypeptides of the present invention can be used as a molecular weight marker on SDS-PAGE gels or on molecular sieve gel filtration columns using art-recognized methods. The polypeptides of the present invention can be used to raise antibodies or to elicit an immune response. The polypeptides can also be used as a reagent, e.g., a labeled reagent, in assays to quantitatively determine levels of the polypeptide or a molecule to which it binds (e.g., a receptor or a ligand) in biological fluids. The polypeptides can also be used as markers for cells or tissues in which the corresponding polypeptide is preferentially expressed, either constitutively, during tissue differentiation, or in a diseased state. The polypeptides can also be used to isolate a corresponding binding agent, and to screen for peptide or small molecule antagonists or agonists of the binding interaction. The polypeptides of the present invention can also be used as therapeutic agents.

Nucleic Acid Molecules of the Invention

The present invention also features isolated seryl tRNA synthetase nucleic acid molecules.

By a “seryl tRNA synthetase nucleic acid molecule” is meant a nucleic acid molecule that encodes a seryl tRNA synthetase polypeptide. Such nucleic acid molecules include, for example, the seryl tRNA synthetase nucleic acid molecule described in detail herein; an isolated nucleic acid comprising SEQ ID NO: 2; a complement of an isolated nucleic acid comprising SEQ ID NO: 2, an isolated nucleic acid encoding a seryl tRNA synthetase polypeptide of SEQ ID NO: 1; a complement of an isolated nucleic acid encoding a seryl tRNA synthetase polypeptide of SEQ ID NO: 1; a nucleic acid that is hybridizable under high stringency conditions to a nucleic acid molecule that encodes SEQ ID NO: 1 or a complement thereof; a nucleic acid molecule that is hybridizable under high stringency conditions to a nucleic acid comprising SEQ ID NO: 2; and an isolated nucleic acid molecule that has at least 55%, more preferably, 60%, 65%, 70%, 75%, 80%, 85%, or 90%, and most preferably, 95% or 99% sequence identity with SEQ ID NO: 2, or a complement thereof. In one embodiment, the percent identity is determined over the full length of the seryl tRNA synthetase gene (e.g., the full length of SEQ ID NO: 2).

The isolated nucleic acid molecules of the present invention can be RNA, for example, mRNA, or DNA, such as cDNA and genomic DNA. DNA molecules can be double-stranded or single-stranded; single stranded RNA or DNA can be either the coding, or sense, strand or the non-coding, or antisense, strand. The nucleic acid molecule can include all or a portion of the coding sequence of the gene and can further comprise additional non-coding sequences such as introns and non-coding 3′ and 5′ sequences (including regulatory sequences, for example). Additionally, the nucleic acid molecule can be fused to a marker sequence, for example, a sequence that encodes a polypeptide to assist in isolation or purification of the polypeptide. Such sequences include, but are not limited to, FLAG tags, as well as sequences that encode a glutathione-S-transferase (GST) fusion protein and those that encode a hemagglutinin A (HA) polypeptide marker from influenza.

An “isolated,” “substantially pure,” or “substantially pure and isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA or cDNA library). For example, an isolated nucleic acid of the invention may be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system, or reagent mix. In other circumstances, the material may be purified to essential homogeneity, for example, as determined by agarose gel electrophoresis or column chromatography such as HPLC. Preferably, an isolated nucleic acid molecule comprises at least about 50, 80, or 90% (on a molar basis) of all macromolecular species present.

With regard to genomic DNA, the term “isolated” also can refer to nucleic acid molecules that are separated from the chromosome with which the genomic DNA is naturally associated. For example, the isolated nucleic acid molecule can contain less than about 5 kb, 4 kb, 3 kb, 2 kb, 1 kb, 0.5 kb, or 0.1 kb of nucleotides that flank the nucleic acid molecule in the genomic DNA of the cell from which the nucleic acid molecule is derived.

The seryl tRNA synthetase nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence that is synthesized chemically or by recombinant means. Therefore, recombinant DNA contained in a vector are included in the definition of “isolated” as used herein.

Isolated nucleotide molecules also include recombinant DNA molecules in heterologous organisms, as well as partially or substantially purified DNA molecules in solution. In vivo and in vitro RNA transcripts of the DNA molecules of the present invention are also encompassed by “isolated” nucleotide sequences. Such isolated nucleotide sequences are useful in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis.

The present invention also pertains to variant seryl tRNA synthetase nucleic acid molecules that are not necessarily found in nature but that encode a seryl tRNA synthetase polypeptide. Thus, for example, DNA molecules that comprise a sequence that is different from the naturally-occurring seryl tRNA synthetase nucleotide sequence but which, due to the degeneracy of the genetic code, encode a seryl tRNA synthetase polypeptide of the present invention are also the subject of this invention.

The invention also encompasses seryl tRNA synthetase nucleotide sequences encoding portions (fragments), or encoding variant polypeptides such as analogues or derivatives of a seryl tRNA synthetase polypeptide. Such variants can be naturally-occurring, such as in the case of allelic variation or single nucleotide polymorphisms, or non-naturally-occurring, such as those induced by various mutagens and mutagenic processes. Intended variations include, but are not limited to, addition, deletion, and substitution of one or more nucleotides that can result in conservative or non-conservative amino acid changes, including additions and deletions. Preferably, the seryl tRNA synthetase nucleotide (and/or resultant amino acid) changes are silent or conserved; that is, they do not alter the characteristics or activity of the seryl tRNA synthetase polypeptide. In one preferred embodiment, the nucleotide sequences are fragments that comprise one or more polymorphic microsatellite markers.

Other alterations of the seryl tRNA synthetase nucleic acid molecules of the invention can include, for example, labeling, methylation, internucleotide modifications such as uncharged linkages (e.g., methyl phosphonates, phosphotriesters, phosphoamidates, and carbamates), charged linkages (e.g., phosphorothioates or phosphorodithioates), pendent moieties (e.g., polypeptides), intercalators (e.g., acridine or psoralen), chelators, alkylators, and modified linkages (e.g., alpha anomeric nucleic acids). Also included are synthetic molecules that mimic nucleic acid molecules in the ability to bind to a designated sequences via hydrogen bonding and other chemical interactions. Such molecules include, for example, those in which peptide linkages substitute for phosphate linkages in the backbone of the molecule.

The invention also pertains to seryl tRNA synthetase nucleic acid molecules that hybridize under high stringency hybridization conditions, such as for selective hybridization, to a nucleotide sequence described herein (e.g., nucleic acid molecules that specifically hybridize to a nucleotide sequence encoding polypeptides described herein, and, optionally, have an activity of the polypeptide). In one embodiment, the invention includes variants described herein that hybridize under high stringency hybridization conditions (e.g., for selective hybridization) to a nucleotide sequence comprising the nucleotide sequence of SEQ ID NO: 2, and the complement of SEQ ID NO: 2. In another embodiment, the invention includes variants described herein that hybridize under high stringency hybridization conditions (e.g., for selective hybridization) to a nucleotide sequence encoding an amino acid sequence of SEQ ID NO: 1. In a preferred embodiment, the variant that hybridizes under high stringency hybridizations encodes a polypeptide that has a biological activity of a seryl tRNA synthetase polypeptide (e.g., seryl tRNA synthetase activity or angiogenic modulatory activity).

Such nucleic acid molecules can be detected and/or isolated by specific hybridization (e.g., under high stringency conditions). “Specific hybridization,” as used herein, refers to the ability of a first nucleic acid to hybridize to a second nucleic acid in a manner such that the first nucleic acid does not hybridize to any nucleic acid other than to the second nucleic acid (e.g., when the first nucleic acid has a higher similarity to the second nucleic acid than to any other nucleic acid in a sample wherein the hybridization is to be performed). “Stringency conditions” for hybridization is a term of art that refers to the incubation and wash conditions, e.g., conditions of temperature and buffer concentration, that permit hybridization of a particular nucleic acid to a second nucleic acid; the first nucleic acid may be perfectly (i.e., 100%) complementary to the second, or the first and second may share some degree of complementarity that is less than perfect (e.g., 70%, 75%, 85%, 95%). For example, certain high stringency conditions can be used that distinguish perfectly complementary nucleic acids from those of less complementarity. “High stringency conditions,” “moderate stringency conditions,” and “low stringency conditions” for nucleic acid hybridizations are explained in Current Protocols in Molecular Biology (See Ausubel et al., supra, the entire teachings of which are incorporated by reference herein). The exact conditions that determine the stringency of hybridization depend not only on ionic strength (e.g., 0.2×SSC or 0.1×SSC), temperature (e.g., room temperature, 42° C. or 68° C.), and the concentration of destabilizing agents such as formamide or denaturing agents such as SDS, but also on factors such as the length of the nucleic acid sequence, base composition, percent mismatch between hybridizing sequences, and the frequency of occurrence of subsets of that sequence within other non-identical sequences. Thus, equivalent conditions can be determined by varying one or more of these parameters while maintaining a similar degree of identity or similarity between the two nucleic acid molecules. Typically, conditions are used such that sequences at least about 60%, at least about 70%, at least about 80%, at least about 90% or at least about 95% or more identical to each other remain hybridized to one another. By varying hybridization conditions from a level of stringency at which no hybridization occurs to a level at which hybridization is first observed, conditions that will allow a given sequence to hybridize (e.g., selectively) with the most similar sequences in the sample can be determined.

Exemplary hybridization conditions are described in Krause and Aaronson, Methods in Enzymology, 200:546-556 (1991), and also in Ausubel, et al., supra, which describes the determination of washing conditions for moderate or low stringency conditions. Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum extent of mismatching among the sequences that hybridize. Generally, doubling the concentration of SSC results in an increase in Tm of 17° C. Using these guidelines, the washing temperature can be determined empirically for high, moderate, or low stringency, depending on the level of mismatch sought.

For example, a low stringency wash can comprise washing in a solution containing 0.2×SSC/0.1% SDS for 10 minutes at room temperature; a moderate stringency wash can comprise washing in a prewarmed solution (42° C.) solution containing 0.2×SSC/0.1% SDS for 15 minutes at 42° C.; and a high stringency wash can comprise washing in prewarmed (68° C.) solution containing 0.1×SSC/0.1% SDS for 15 minutes at 68° C. Furthermore, washes can be performed repeatedly or sequentially to obtain a desired result as known in the art. Equivalent conditions can be determined by varying one or more of the parameters given as an example, as known in the art, while maintaining a similar degree of identity or similarity between the target nucleic acid molecule and the primer or probe used.

The present invention also provides isolated seryl tRNA synthetase nucleic acid molecules that contain a fragment or portion that hybridizes under highly stringent conditions to a nucleotide sequence comprising a nucleotide sequence selected from SEQ ID NO: 2, and the complement of SEQ ID NO: 2, and also provides isolated nucleic acid molecules that contain a fragment or portion that hybridizes under highly stringent conditions to a nucleotide sequence encoding an amino acid sequence selected from SEQ ID NO: 1. The nucleic acid fragments of the invention are at least about 15, preferably, at least about 18, 20, 23, or 25 nucleotides, and can be 30, 40, 50, 100, 200 or more nucleotides in length. Fragments that are, for example, 30 or more nucleotides in length, that encode antigenic polypeptides described herein are particularly useful, such as for the generation of antibodies as described above.

In a related aspect, the seryl tRNA synthetase nucleic acid fragments of the invention are used as probes or primers in assays such as those described herein. “Probes” or “primers” are oligonucleotides that hybridize in a base-specific manner to a complementary strand of nucleic acid molecules. Such probes and primers include polypeptide nucleic acids, as described in Nielsen et al., Science, 254, 1497-1500 (1991). As also used herein, the term “primer” in particular refers to a single-stranded oligonucleotide that acts as a point of initiation of template-directed DNA synthesis using well-known methods (e.g., PCR, LCR) including, but not limited to those described herein.

Typically, a probe or primer comprises a region of nucleotide sequence that hybridizes to at least about 15, typically about 20-25, and more typically about 40, 50 or 75, consecutive nucleotides of a nucleic acid molecule comprising a contiguous nucleotide sequence selected from: SEQ ID NO: 2, the complement of SEQ ID NO: 2, and a sequence encoding an amino acid sequence of SEQ ID NO: 1.

In preferred embodiments, a probe or primer comprises 100 or fewer nucleotides, preferably, from 6 to 50 nucleotides, and more preferably, from 12 to 30 nucleotides. In other embodiments, the probe or primer is at least 70% identical to the contiguous nucleotide sequence or to the complement of the contiguous nucleotide sequence, preferably, at least 80% identical, more preferably, at least 90% identical, even more preferably, at least 95% identical, or even capable of selectively hybridizing to the contiguous nucleotide sequence or to the complement of the contiguous nucleotide sequence. Often, the probe or primer further comprises a label, e.g., radioisotope, fluorescent compound, enzyme, or enzyme co-factor.

The nucleic acid molecules of the invention such as those described above can be identified and isolated using standard molecular biology techniques and the sequence information provided in SEQ ID NO: 1, and/or SEQ ID NO: 2. For example, nucleic acid molecules can be amplified and isolated by the polymerase chain reaction using synthetic oligonucleotide primers designed based on one or more of the nucleic acid sequences provided above and/or the complement of those sequences. Or such nucleic acid molecules may be designed based on nucleotide sequences encoding the amino acid sequences provided in SEQ ID NO: 1. See generally PCR Technology: Principles and Applications for DNA Amplification (ed. H. A. Erlich, Freeman Press, New York, N.Y., (1992); PCR Protocols: A Guide to Methods and Applications (Eds. Innis et al., Academic Press, San Diego, Calif., (1990); Mattila et al., Nucleic Acids Res., 19: 4967 (1991); Eckert et al., PCR Methods and Applications, 1:17 (1991); PCR (eds. McPherson et al., IRL Press, Oxford)); and U.S. Pat. No. 4,683,202. The nucleic acid molecules can be amplified using cDNA, mRNA, or genomic DNA as a template, cloned into an appropriate vector and characterized by DNA sequence analysis.

Other suitable amplification methods include the ligase chain reaction (LCR) (See Wu and Wallace, Genomics, 4:560 (1989); and Landegren et al., Science, 241:1077 (1988)), transcription amplification (Kwoh et al., Proc. Natl. Acad. Sci. USA, 86:1173 (1989)), and self-sustained sequence replication (See Guatelli et al., Proc. Nat. Acad. Sci. USA, 87:1874 (1990)) and nucleic acid based sequence amplification (NASBA). The latter two amplification methods involve isothermal reactions based on isothermal transcription, that produce both single stranded RNA (ssRNA) and double stranded DNA (dsDNA) as the amplification products in a ratio of about 30 or 100 to 1, respectively.

The amplified DNA can be radiolabeled and used as a probe for screening a cDNA library, for example, one derived from human cells or any other desired cell type. Corresponding clones can be isolated, DNA can be obtained following in vivo excision, and the cloned insert can be sequenced in either or both orientations by art-recognized methods to identify the correct reading frame encoding a polypeptide of the appropriate molecular weight. For example, the direct analysis of the nucleotide sequence of nucleic acid molecules of the present invention can be accomplished using well-known methods that are commercially available. See, for example, Sambrook et al., Molecular Cloning, A Laboratory Manual (2nd Ed., CSHP, New York (1989)); and Zyskind et al., Recombinant DNA Laboratory Manual, (Acad. Press, (1988)). Using these or similar methods, the polypeptide and the DNA encoding the polypeptide can be isolated, sequenced, and further characterized.

Antisense nucleic acid molecules of the invention can be designed using the nucleotide sequence of SEQ ID NO: 2, and/or the complement of SEQ ID NO: 2, and/or a portion of those sequences, and/or the complement of those portions or sequences, and/or a sequence encoding the amino acid sequence of SEQ ID NO: 1, or encoding a portion of SEQ ID NO: 1. The methods are based on binding of a polynucleotide to a complementary DNA or RNA. In one embodiment, an antisense sequence is generated internally by the organism, in another embodiment, the antisense sequence is separately administered (see, for example, O'Connor, J. Neurochem. 56:560 (1991)).

In one embodiment, the 5′ coding portion of an informative gene can be used to design an antisense RNA oligonucleotide from about 10 to 40 base pairs in length. Generally, a DNA oligonucleotide is designed to be complementary to a region of the gene involved in transcription thereby preventing transcription and the production of the receptor. The antisense RNA oligonucleotide hybridizes to the mRNA in vivo and blocks translation of the mRNA molecule into receptor polypeptide.

In one embodiment, the antisense nucleic acid of the invention is produced intracellularly by transcription from an exogenous sequence. For example, a vector or a portion thereof, is transcribed, producing an antisense nucleic acid of the invention. Such a vector contains the sequence encoding the antisense nucleic acid. The vector can remain episomal or become chromosomally integrated, as long as it can be transcribed to produce the desired antisense RNA. Vectors can be constructed by recombinant DNA technology and can be plasmid, viral, or otherwise, as is known to one of skill in the art.

Expression can be controlled by any promoter or functional part of a promoter known in the art to act in the target cells, such as vertebrate cells, and preferably human cells. Such promoters can be inducible or constitutive and include, without limitation, the SV40 early promoter region (Bernoist and Chambon, Nature 29:304-310(1981), the promoter contained in the 3′ long terminal repeat of Rous sarcoma virus (Yamamoto et al., Cell 22:787-797 (1980)), the herpes thymidine promoter (Wagner et al., Proc. Natl. Acad. Sci. U.S.A. 78:1441-1445 (1981)), and the regulatory sequences of the metallothionein gene (Brinster et al., Nature 296:39-42 (1982)). A functional part of a promoter can be identified, for example, by generating promoter fragments, and testing the promoter fragments in a reporter gene assay, described, for example, in Ausubel et al (supra). Those promoter fragments that retain promoter activity when compared to the full length promoter are function promoter fragments.

Alternatively, the promoter, or functional part of a promoter, that is naturally associated with the seryl tRNA synthetase gene can be used to promoter expression. Methods for cloning promoter regions of genes are known in the art, and are described, for example, in Ausubel et al. (supra).

The antisense nucleic acids of the invention comprise a sequence complementary to at least a nortion of an RNA transcript of an informative gene. Absolute complementarity, although preferred, is not required. A sequence “complementary to at least a portion of an RNA,” referred to herein, means a sequence having sufficient complementarity to be able to hybridize with the RNA, forming a stable duplex. The ability to hybridize will depend on both the degree of complementarity and the length of the antisense nucleic acid. Generally, the larger the hybridizing nucleic acid, the more base mismatches with the RNA it may contain and still form a stable duplex. One skilled in the art can ascertain a tolerable degree of mismatch by use of standard procedures to determine the melting point of the hybridized complex.

Oligonucleotides that are complementary to the 5′ end of the RNA, for example, the 5′ untranslated sequence up to and including the AUG initiation codon, are generally regarded to work most efficiently at inhibiting translation. However, sequences complementary to the 3′ untranslated sequences of mRNAs have been shown to be effective at inhibiting translation of mRNAs as well. Thus, oligonucleotides complementary to either the 5′- or 3′-non-translated, non-coding regions of a nucleotide sequence can be used in an antisense approach to inhibit mRNA translation. Oligonucleotides complementary to the 5′ untranslated region of the mRNA can include the complement of the AUG start codon. Antisense oligonucleotides complementary to mRNA coding regions can also be used in accordance with the invention.

The antisense oligonucleotides of the invention can be DNA or RNA, or chimeric mixtures, or derivatives or modified versions thereof, single-stranded or double-stranded. The oligonucleotide can be modified at the base moiety, sugar moiety, or phosphate backbone, for example, to improve stability of the molecule, hybridization, and the like. The oligonucleotide can include other appended groups such as peptides (for example, to target host cell receptors in vivo), or agents that facilitate transport across the cell membrane, or the blood-brain barrier, or intercalating agents.

The antisense oligonucleotide may comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, a-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including, but not limited to, arabinose, 2-fluoroarabinose, xylulose, and hexose.

In yet another embodiment, the antisense oligonucleotide comprises at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., Nucl. Acids Res. 15:6625-6641 (1987)). The oligonucleotide is a 2′-O-methylribonucleotide (Inoue et al., Nucl. Acids Res. 15:6131-6148 (1987)), or a chimeric RNA-DNA analog (Inoue et al., FEBS Lett. 215:327-330 (1987)).

Antisense oligonucleotides of the invention may be synthesized by standard methods known in the art, for example, by use of an automated DNA synthesizer.

Potential antagonists according to the invention also include catalytic RNA, or a ribozyme. Hammerhead ribozymes cleave mRNAs at locations dictated by flanking regions that form complementary base pairs with the target mRNA. The target mRNA has the following sequence of two bases: 5′-UG-3′. The construction and production of hammerhead ribozymes is well known in the art and is described more fully in Haseloff and Gerlach (Nature 334:585-591 (1988)). Preferably, the ribozyme is engineered so that the cleavage recognition site is located near the 5′ end of the mRNA in order to increase efficiency and minimize the intracellular accumulation of non-functional mRNA transcripts.

Ribozymes of the invention can be composed of modified oligonucleotides (for example for improved stability, targeting, and the like). DNA constructs encoding the ribozyme can be under the control of a strong constitutive promoter, such as, for example, pol III or pol II promoter, so that a transfected cell will produce sufficient quantities of the ribozyme to destroy endogenous target mRNA and inhibit translation. Since ribozymes, unlike antisense molecules, are catalytic, a lower intracellular concentration is generally required for efficiency.

In general, the isolated seryl tRNA synthetase nucleic acid sequences of the invention can be used as molecular weight markers on Southern blots, and as chromosome markers that are labeled to map related gene positions. The nucleic acid sequences can also be used to compare with endogenous DNA sequences in individuals to identify genetic disorders (e.g., a predisposition for or susceptibility to an angiogenic disease, a vascular disease, a heart disease, or a circulatory disease, and as probes, such as to hybridize and discover related DNA sequences or to subtract out known sequences from a sample. The nucleic acid molecules of the present invention can also be used as therapeutic agents.

By an “angiogenic disease” is meant a disease that is caused by or results in excessive (abnormally or undesirably high) levels of blood vessel formation, insufficient (abnormally or undesirably low) levels of blood vessel formation, or blood vessel formation in an area of the body where it normally does not occur. Excessive angiogenesis occurs in diseases such as cancer, diabetic blindness, age-related macular degeneration, rheumatoid arthritis, and psoriasis, and more than 70 other conditions. In these conditions, new blood vessels feed diseased tissues, destroy normal tissues, and in the case of cancer, the new vessels allow tumor cells to escape into the circulation and lodge in other organs (tumor metastases). Excessive angiogenesis occurs, for example, when diseased cells produce abnormal amounts of angiogenic growth factors, overwhelming the effects of natural angiogenesis inhibitors. Insufficient angiogenesis occurs in diseases such as coronary artery disease, stroke, and delayed wound healing. In these conditions, inadequate blood vessels grow, and circulation is not properly restored, leading to the risk of tissue death. Insufficient angiogenesis occurs, for example, when the tissue cannot produce adequate amounts of angiogenic growth factors.

By a “vascular disease” is meant a disease that is characterized by abnormal formation or function of vasculature. The vascular disease can be cause by excessive formation of blood vessels, by insufficient formation of blood vessels, or by a vasodilation, vasorelaxation, or vasoconstriction, resulting in altered blood flow. Examples of vascular diseases include coronary artery disease, stroke, delayed wound healing, cancer, diabetic blindness, age-related macular degeneration, rheumatoid arthritis, and psoriasis.

By a “heart disease” is meant a condition in which the heart and/or vasculature leading to or away from the heart has altered formation or function. In one embodiment, the altered formation involves the vasculature that connects the heart to the rest of the circulatory system. In another embodiment, the heart disease is caused by a vascular disease.

By a “circulatory disease” is meant a condition characterized by increased or decreased circulation of blood throughout the body. In one embodiment, the circulatory disease is a decrease (for example, 90%, 80%, 70%, 60%, 50%, 40%, 30%, 20%, 10%, or 5% of the blood flow through all or a part of the body compared to a healthy normal individual) or complete cessation of circulation. Such a disease can be caused, for example, by a vascular disease or a heart disease, as described herein.

The seryl tRNA synthetase nucleic acid molecules of the present invention can further be used to derive primers for genetic fingerprinting, to raise anti-polypeptide antibodies using DNA immunization techniques, and as an antigen to raise anti-DNA antibodies or elicit immune responses. Portions or fragments of the nucleotide sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample.

In addition, the seryl tRNA synthetase nucleotide sequences of the invention can be used to identify and express recombinant polypeptides for analysis, characterization, or therapeutic use, or as markers for tissues in which the corresponding polypeptide is expressed, either constitutively, during tissue differentiation, or in diseased states. The nucleic acid sequences can additionally be used as reagents in the screening and/or diagnostic assays described herein, and can also be included as components of kits (e.g., reagent kits) for use in the screening and/or diagnostic assays described herein.

Standard techniques, such as the polymerase chain reaction (PCR) and DNA hybridization, may be used to clone seryl tRNA synthetase homologs in other species, for example, mammalian homologs. Seryl tRNA synthetase homologs may be readily identified using low-stringency DNA hybridization or low-stringency PCR with human seryl tRNA synthetase probes or primers. Degenerate primers encoding human seryl tRNA synthetase polypeptides may be used to clone seryl tRNA synthetase homologs by RT-PCR.

Alternatively, additional seryl tRNA synthetase homologs can be identified by utilizing consensus sequence information for seryl tRNA synthetase polypeptides to search for similar polypeptides in other species. For example, polypeptide databases for other species can be searched for proteins with the seryl tRNA synthetase domains described herein. Candidate polypeptides containing such a motif can then be tested for their seryl tRNA synthetase biological activities, using methods described herein.

Expression of the Nucleic Acid Molecules of the Invention

Another aspect of the invention pertains to nucleic acid constructs containing a seryl tRNA synthetase, nucleic acid molecule, for example, one selected from the group consisting of SEQ ID NO: 2, and the complement of any of SEQ ID NO: 2 (or portions thereof). Yet another aspect of the invention pertains to seryl tRNA synthetase nucleic acid constructs containing a nucleic acid molecule encoding the amino acid sequence of SEQ ID NO: 1. The constructs comprise a vector (e.g., an expression vector) into which a sequence of the invention has been inserted in a sense or antisense orientation.

As used herein, the term “vector” or “construct” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids. However, the invention is intended to include such other forms of expression vectors, such as viral vectors (e.g., replication defective retroviruses, adenoviruses and adeno-associated viruses) that serve equivalent functions.

Preferred recombinant expression vectors of the invention comprise a nucleic acid molecule of the invention in a form suitable for expression of the nucleic acid molecule in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner that allows for expression of the nuleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences).

It will be appreciated by those skilled in the art that the design of the expression vector can depend on such factors as the choice of the host cell to be transformed and the level of expression of polypeptide desired. The expression vectors of the invention can be introduced into host cells to thereby produce polypeptides, including fusion polypeptides, encoded by nucleic acid molecules as described herein.

The recombinant expression vectors of the invention can be designed for expression of a polypeptide of the invention in prokaryotic or eukaryotic cells, e.g., bacterial cells, such as E. coli, insect cells (using baculovirus expression vectors), yeast cells or mammalian cells. Suitable host cells are discussed further in Goeddel, supra. Alternatively, the recombinant expression vector can be transcribed and translated in vitro, for example, using T7 promoter regulatory sequences and T7 polymerase.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a nucleic acid molecule of the invention can be expressed in bacterial cells (e.g., E. coli), insect cells, yeast, or mammalian cells (such as Chinese hamster ovary cells (CHO) or COS cells, human 293T cells, HeLa cells, NIH 3T3 cells, and mouse erythroleukemia (MEL) cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing a foreign nucleic acid molecule (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those that confer resistance to drugs, such as G418, hygromycin, or methotrexate. Nucleic acid molecules encoding a selectable marker can be introduced into a host cell on the same vector as the nucleic acid molecule of the invention or can be introduced on a separate vector. Cells stably transfected with the introduced nucleic acid molecule can be identified by drug selection (e.g., cells that have incorporated the selectable marker gene will survive, while the other cells die).

A host cell of the invention, such as a prokaryotic or eukaryotic host cell in culture, can be used to produce (i.e., express) a polypeptide of the invention. Accordingly, the invention further provides methods for producing a polypeptide using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

The host cells of the invention can also be used to produce nonhuman transgenic animals. For example, in one embodiment, a host cell of the invention is a fertilized oocyte or an embryonic stem cell into which a seryl tRNA synthetase nucleic acid molecule of the invention has been introduced. Such host cells can then be used to create non-human transgenic animals in which exogenous nucleotide sequences have been introduced into the genome or homologous recombinant animals in which endogenous nucleotide sequences have been altered. Such animals are useful for studying the function and/or activity of the nucleotide sequence and polypeptide encoded by the sequence and for identifying and/or evaluating modulators of their activity.

As used herein, a “transgenic animal” is a non-human animal, preferably, a vertebrate, for example a fish (e.g., a zebrafish), a mammal, for example, a rodent such as a rat or mouse, in which one or more of the cells of the animal includes a transgene. Other examples of transgenic animals include non-human primates, sheep, dogs, cows, goats, chickens, and amphibians. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably, a mammal, more preferably, a mouse, in which an endogenous gene has been altered by homologous recombination between the endogenous gene and an exogenous DNA molecule introduced into a cell of the animal, e.g., an embryonic cell of the animal, prior to development of the animal.

Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191, and in Hogan, Manipulating the Mouse Embryo (Cold Spring Harbor Laboratory Press, Cold Spring Harbor, N.Y., (1986)). Methods for constructing homologous recombination vectors and homologous recombinant animals are described further in Bradley, Current Opinion in Bio/Technology, 2:823-829 (1991) and in PCT Publication Nos. WO 90/11354, WO 91/01140, WO 92/0968, and WO 93/04169. Clones of the non-human transgenic animals described herein can also be produced according to the methods described in Wilmut et al., Nature, 385:810-813 (1997) and PCT Publication Nos. WO 97/07668 and WO 97/07669. Methods for generating transgenic zebrafish are also known in the art.

Antibodies of the Invention

Polyclonal and/or monoclonal antibodies that selectively bind a seryl tRNA synthetase polypeptide are also provided. Antibodies are also provided that bind a portion of either a variant or reference seryl tRNA synthetase polypeptide that contains a polymorphic site or sites.

In another aspect, the invention provides antibodies to a seryl tRNA synthetase polypeptide or polypeptide fragment of the invention, e.g., having an amino acid sequence encoded by SEQ ID NO: 1, or a portion thereof, or having an amino acid sequence encoded by a nucleic acid molecule comprising all or a portion of SEQ ID NO: 2, or another variant, or portion thereof.

The term “purified antibody” as used herein refers to immunoglobulin molecules and immunologically active portions of immunoglobulin molecules, i.e., molecules that contain an antigen binding site that selectively binds an antigen. A molecule that selectively binds to a polypeptide of the invention is a molecule that binds to that polypeptide or a fragment thereof, but does not substantially bind other molecules in a sample, e.g., a biological sample that naturally contains the polypeptide. Preferably the antibody is at least 60%, by weight, free from proteins and naturally occurring organic molecules with which it naturally associated. More preferably, the antibody preparation is at least 75% or 90%, and most preferably, 99%, by weight, antibody. Examples of immunologically active portions of immunoglobulin molecules include F(ab) and F(ab′)2 fragments that can be generated by treating the antibody with an enzyme such as pepsin.

The invention provides polyclonal and monoclonal antibodies that selectively hind to a seryl tRNA synthetase polypeptide of the invention. The term “monoclonal antibody” or “monoclonal antibody composition,” as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., a seryl tRNA synthetase polypeptide of the invention or fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction.

At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4:72 (1983)), the EBV-hybridoma technique (Cole et al., Monoclonal Antibodies and Cancer Therapy, Alan R. Liss, Inc., pp. 77-96 (1985)) or trioma techniques. The technology for producing hybridomas is well known (see generally Current Protocols in Immunology, Coligan et al., (eds.) John Wiley & Sons, Inc., New York, N.Y. (1994)). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfe et al., Nature, 266:55052 (1977); R. H. Kenneth, in Monoclonal Antibodies: A New Dimension In Biological Analyses, Plenum Publishing Corp., New York, N.Y. (1980); and Lemer, Yale J. Biol. Med. 54:387-402 (1981)). Moreover, the ordinarily skilled worker will appreciate that there are many variations of such methods that also would be useful.

In one alternative to preparing monoclonal antibody-secreting hybridomas, a monoclonal antibody to a seryl tRNA synthetase polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9:1370-1372 (1991); Hay et al., Hum. Antibod. Hybridomas 3:81-85 (1992); Huse et al., Science 246:1275-1281 (1989); and Griffiths et al., EMBO J. 12:725-734 (1993).

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a seryl tRNA synthetase polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a seryl tRNA synthetase polypeptide of the invention can be used to detect the polypeptide (e.g., in a cellular lysate, cell supernatant, or tissue sample) in order to evaluate the abundance and pattern of expression of the polypeptide.

The antibodies of the present invention can also be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, and acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride and phycoerythrin; an example of a luminescent material includes luminol; examples of bioluminescent materials include luciferase, luciferin, green fluorescent protein, and aequorin, and examples of suitable radioactive material include, for example, ¹²⁵I, ¹³¹I, ³⁵S, ³²P and ³H.

Zebrafish with a Mutated Seryl Transfer RNA Synthetase Gene

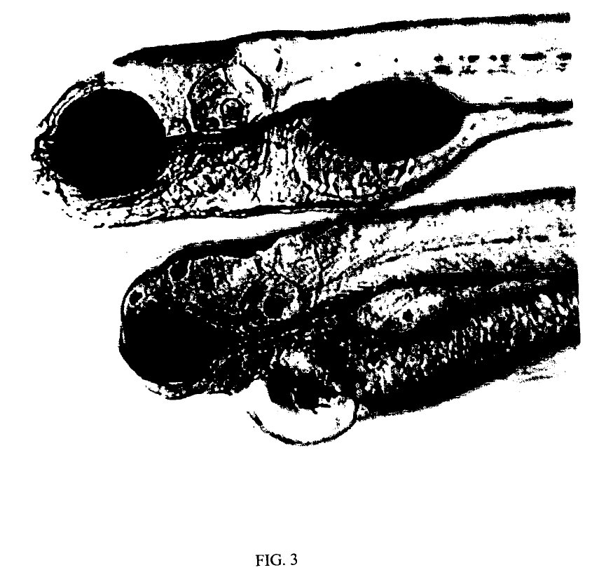

The invention also features zebrafish having a mutated tRNA synthetase gene. The fish have a phenotype whereby blood circulates through the heart and a portion of the head, but does not circulate thought the trunk (FIG. 3). The phenotype is observed by within 5 days post-fertilization, and may be caused by altered angiogenic activity, a heart condition, or vascular disease. Expression of the seryl tRNA synthetase gene is greatly reduced in the mutant fish due to interruption of the gene by a proviral insert in an intron of the gene. This proviral insert is located in an intron of the DNA located after nucleotide 168 of the cDNA sequence of FIG. 2. The generation of such a mutant zebrafish is described in detail herein. These mutant zebrafish and their wild-type counterparts (i.e., not having a mutated seryl tRNA synthetase gene) can be used, for example, to better understand vasculature development and angiogenesis, and as reagents in screening methods to identify compounds that can be used to modulate seryl tRNA synthetase biological activities, and to treat an angiogenic disease, a heart disease, a circulatory disease, or a vascular disease, as described herein.

Diagnostic and Screening Assays of the Invention

The present invention also pertains to diagnostic assays for assessing seryl tRNA synthetase gene expression, or for assessing activity of seryl tRNA synthetase polypeptides of the invention. In one embodiment, the assays are used in the context of a biological sample (e.g., blood, serum, cells, tissue) to thereby determine whether an individual is afflicted with an angiogenic disease, a vascular disease, a heart disease, or a circulatory disease, or is at risk for (has a predisposition for or a susceptibility to) developing an angiogenic disease, a vascular disease, a heart disease, or a circulatory disease. The invention also provides for prognostic (or predictive) assays for determining whether an individual is susceptible to developing an angiogenic disease, a vascular disease, a heart disease, or a circulatory disease. For example, mutations in the seryl tRNA synthetase nucleic acid molecule can be assayed in a biological sample. Such assays can be used for prognostic or predictive purpose to thereby prophylactically treat an individual prior to the onset of symptoms associated with an angiogenic disease, a vascular disease, a heart disease, or a circulatory disease.

Another aspect of the invention pertains to assays for monitoring the influence of agents, or candidate compounds (e.g., drugs or other agents) on the nucleic acid molecule expression or biological activity of polypeptides of the invention, as well as to assays for identifying candidate compounds that bind to a seryl tRNA synthetase polypeptide. These and other assays and agents are described in further detail in the following sections.

Diagnostic Assays

Seryl tRNA synthetase nucleic acid molecules, probes, primers, polypeptides, and antibodies to a seryl tRNA synthetase protein can be used in methods of diagnosis of a susceptibility to, or likelihood of having an angiogenic disease, a vascular disease, a heart disease, or a circulatory disease, as well as in kits useful for diagnosis of a susceptibility to an angiogenic disease, a vascular disease, a heart disease, or a circulatory disease.

In one embodiment of the invention, diagnosis of an altered (i.e., increased or decreased) susceptibility to an angiogenic disease, a vascular disease, a heart disease, or a circulatory disease is made by detecting a polymorphism in seryl tRNA synthetase. The polymorphism can be a mutation in seryl tRNA synthetase, such as the insertion or deletion of a single nucleotide, or of more than one nucleotide, resulting in a frame shift mutation; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of the gene; duplication of all or a part of the gene; transposition of all or a part of the gene; or rearrangement of all or a part of the gene. More than one such mutation may be present in a single nucleic acid molecule.

Such sequence changes cause a mutation in the polypeptide encoded by seryl tRNA synthetase. For example, if the mutation is a frame shift mutation, the frame shift can result in a change in the encoded amino acids, and/or can result in the generation of a premature stop codon, causing generation of a truncated polypeptide. Alternatively, a polymorphism associated with an altered susceptibility to an angiogenic disease, a vascular disease, a heart disease, or a circulatory disease can be a synonymous mutation in one or more nucleotides (i.e., a mutation that does not result in a change in the seryl tRNA synthetase. Such a polymorphism may alter sites, affect the stability or transport of mRNA, or otherwise affect the transcription or translation of the nucleic acid molecule. Seryl tRNA synthetase that has any of the mutations described above is referred to herein as a “mutant nucleic acid molecule.”

In a first method of diagnosing an altered susceptibility to an angiogenic disease, a vascular disease, a heart disease, or a circulatory disease, hybridization methods, such as Southern analysis, Northern analysis, or in situ hybridizations, can be used (see Ausubel, et al., supra). For example, a biological sample from a test subject (a “test sample”) of genomic DNA, RNA, or cDNA, is obtained from an individual suspected of having, being susceptible to or predisposed for, or carrying a defect for, an angiogenic disease, a vascular disease, a heart disease, or a circulatory disease (the “test individual”). The individual can be an adult, child, or fetus. The test sample can be from any source that contains genomic DNA, such as a blood sample, sample of amniotic fluid, sample of cerebrospinal fluid, or tissue sample from skin, muscle, buccal or conjunctival mucosa, placenta, gastrointestinal tract, or other organs. A test sample of DNA from fetal cells or tissue can be obtained by appropriate methods, such as by amniocentesis or chorionic villus sampling. The DNA, RNA, or cDNA sample is then examined to determine whether a polymorphism in seryl tRNA synthetase is present, and/or to determine which variant(s) encoded by seryl tRNA synthetase is present. The presence of the polymorphism or variant(s) can be indicated by hybridization of the gene in the genomic DNA, RNA, or cDNA to a nucleic acid probe. A “nucleic acid probe,” as used herein, can be a DNA probe or an RNA probe; the nucleic acid probe can contain at least one polymorphism in seryl tRNA synthetase or contains a nucleic acid encoding a particular variant of seryl tRNA synthetase. The probe can be any of the nucleic acid molecules described above (e.g., the entire nucleic acid molecule, a fragment, a vector comprising the gene, a probe, or primer, etc.).

To diagnose an altered susceptibility to an angiogenic disease, a vascular disease, a heart disease, or a circulatory disease, a hybridization sample is formed by contacting the test sample containing seryl tRNA synthetase, with at least one nucleic acid probe. A preferred probe for detecting mRNA or genomic DNA is a labeled nucleic acid probe capable of hybridizing to seryl tRNA synthetase mRNA or genomic DNA sequences described herein. The nucleic acid probe can be, for example, a full-length nucleic acid molecule, or a portion thereof, such as an oligonucleotide of at least 15, 30, 50, 100, 250, or 500 nucleotides in length and sufficient to specifically hybridize under stringent conditions to appropriate mRNA or genomic DNA. For example, the nucleic acid probe can be all or a portion of SEQ ID NO: 2, or the complement of SEQ ID NO: 2; or can be a nucleic acid molecule encoding all or a portion of SEQ ID NO: 1. Other suitable probes for use in the diagnostic assays of the invention are described above (see. e.g., probes and primers discussed under the heading, “Nucleic Acids of the Invention”).

The hybridization sample is maintained under conditions that are sufficient to allow specific hybridization of the nucleic acid probe to seryl tRNA synthetase. “Specific hybridization,” as used herein, indicates exact hybridization (e.g., with no mismatches). Specific hybridization can be performed under high stringency conditions or moderate stringency conditions, for example, as described above. In a particularly preferred embodiment, the hybridization conditions for specific hybridization are high stringency.

Specific hybridization, if present, is then detected using standard methods. If specific hybridization occurs between the nucleic acid probe and seryl tRNA synthetase in the test sample, then seryl tRNA synthetase has the polymorphism, or is the variant, that is present in the nucleic acid probe. More than one nucleic acid probe can also be used concurrently in this method. Specific hybridization of any one of the nucleic acid probes is indicative of a polymorphism in seryl tRNA synthetase, or of the presence of a particular variant encoded by seryl tRNA synthetase, and is therefore diagnostic for an altered susceptibility to an angiogenic disease, a vascular disease, a heart disease, or a circulatory disease.

In Northern analysis (see Current Protocols in Molecular Biology, Ausubel, et al., supra), the hybridization methods described above are used to identify the presence of a polymorphism or of a particular variant, associated with an altered susceptibility to an angiogenic disease, a vascular disease, a heart disease, or a circulatory disease. For Northern analysis, a test sample of RNA is obtained from the individual by appropriate means. Specific hybridization of a nucleic acid probe, as described above, to RNA from the individual is indicative of a polymorphism in seryl tRNA synthetase, or of the presence of a particular variant encoded by seryl tRNA synthetase, and is therefore diagnostic for an altered susceptibility to an angiogenic disease, a vascular disease, a heart disease, or a circulatory disease.

For representative examples of use of nucleic acid probes, see, for example, U.S. Pat. Nos. 5,288,611 and 4,851,330.

Alternatively, a peptide nucleic acid (PNA) probe can be used instead of a nucleic acid probe in the hybridization methods described above. PNA is a DNA mimic having a peptide-like, inorganic backbone, such as N-(2-aminoethyl)glycine units, with an organic base (A, G, C, T, or U) attached to the glycine nitrogen via a methylene carbonyl linker (see, for example, Nielsen et al., Bioconjugate Chemistry, 5 (1994), American Chemical Society, p. 1 (1994)). The PNA probe can be designed to specifically hybridize to a gene having a polymorphism associated with a susceptibility to an angiogenic disease, a vascular disease, a heart disease, or a circulatory disease. Hybridization of the PNA probe to seryl tRNA synthetase is diagnostic for an altered susceptibility to an angiogenic disease, a vascular disease, a heart disease, or a circulatory disease.

In another method of the invention, mutation analysis by restriction digestion can be used to detect a mutant nucleic acid molecule, or nucleic acid molecules containing a polymorphism(s), if the mutation or polymorphism in the gene results in the creation or elimination of a restriction site. A test sample containing genomic DNA is obtained from the individual. Polymerase chain reaction (PCR) can be used to amplify seryl tRNA synthetase (and, if necessary, the flanking sequences) in the test sample of genomic DNA from the test individual. RFLP analysis is conducted as described (see Current Protocols in Molecular Biology, supra). The digestion pattern of the relevant DNA fragment indicates the presence or absence of the mutation or polymorphism in seryl tRNA synthetase, and therefore indicates the presence or absence of this altered susceptibility to an angiogenic disease, a vascular disease, a heart disease, or a circulatory disease.

Sequence analysis can also be used to detect specific polymorphisms in seryl tRNA synthetase. A test sample of DNA or RNA is obtained from the test individual. PCR or other appropriate methods can be used to amplify the nucleic acid molecule, and/or its flanking sequences, if desired. The sequence of seryl tRNA synthetase, or a fragment thereof, or a seryl tRNA synthetase cDNA, or a fragment thereof, or a seryl tRNA synthetase mRNA, or a fragment thereof, is determined, using standard methods. The sequence of the above gene, gene fragment, cDNA, cDNA fragment, mRNA, or mRNA fragment is compared with the known nucleic acid sequence of the nucleic acid molecule, cDNA (e.g., SEQ ID NO: 2, or a nucleic acid sequence encoding the protein of SEQ ID NO: 1, or a fragment thereof) or mRNA, as appropriate. The presence of a polymorphism in seryl tRNA synthetase indicates that the individual has an altered susceptibility to an angiogenic disease, a vascular disease, a heart disease, or a circulatory disease.

Allele-specific oligonucleotides can also be used to detect the presence of a polymorphism in seryl tRNA synthetase, through the use of dot-blot hybridization of amplified oligonucleotides with allele-specific oligonucleotide (ASO) probes (see, for example, Saiki et al., Nature (London) 324:163-166 (1986)). An “allele-specific oligonucleotide” (also referred to herein as an “allele-specific oligonucleotide probe”) is an oligonucleotide of approximately 10-50 base pairs, preferably approximately 15-30 base pairs, that specifically hybridizes to seryl tRNA synthetase, and that contains a polymorphism associated with an altered susceptibility to an angiogenic disease, a vascular disease, a heart disease, or a circulatory disease. An allele-specific oligonucleotide probe that is specific for particular polymorphisms in seryl tRNA synthetase can be prepared, using standard methods (see Current Protocols in Molecular Biology, supra).

To identify polymorphisms in the gene that are associated with an altered susceptibility to an angiogenic disease, a vascular disease, a heart disease, or a circulatory disease a test sample of DNA is obtained from the individual. PCR can be used to amplify all or a fragment of seryl tRNA synthetase, and its flanking sequences. The DNA containing the amplified seryl tRNA synthetase (or a fragment of the gene) is dot-blotted, using standard methods (see Current Protocols in Molecular Biology, supra), and the blot is contacted with the oligonucleotide probe. The presence of specific hybridization of the probe to the amplified seryl tRNA synthetase is then detected. Specific hybridization of an allele-specific oligonucleotide probe to DNA from the individual is indicative of a polymorphism in seryl tRNA synthetase, and is therefore indicative of an altered susceptibility to an angiogenic disease, a vascular disease, a heart disease, or a circulatory disease.

In another embodiment, arrays of oligonucleotide probes that are complementary to target nucleic acid sequence segments from an individual, can be used to identify polymorphisms in seryl tRNA synthetase. For example, in one embodiment, an oligonucleotide array can be used. Oligonucleotide arrays typically comprise a plurality of different oligonucleotide probes that are coupled to a surface of a substrate in different known locations. These oligonucleotide arrays, also described as “GENECHIPS™,” have been generally described in the art, for example, U.S. Pat. No. 5,143,854 and PCT patent publication Nos. WO 90/15070 and 92/10092. These arrays can generally be produced using mechanical synthesis methods or light directed synthesis methods that incorporate a combination of photolithographic methods and solid phase oligonucleotide synthesis methods. See Fodor et al., Science 251:767-777 (1991), U.S. Pat. No. 5,143,854; PCT Publication No. WO 90/15070; PCT Publication No. WO 92/10092, and U.S. Pat. No. 5,424,186, the entire teachings of each of which are incorporated by reference herein. Techniques for the synthesis of these arrays using mechanical synthesis methods are described in, e.g., U.S. Pat. No. 5,384,261, the entire teachings of which are incorporated by reference herein.

Once an oligonucleotide array is prepared, a nucleic acid of interest is hybridized to the array and scanned for polymorphisms. Hybridization and scanning are generally carried out by methods described herein and also in, e.g., PCT Publication Nos. WO 92/10092 and WO 95/11995, and U.S. Pat. No. 5,424,186, the entire teachings of which are incorporated by reference herein. In brief, a target nucleic acid sequence that includes one or more previously identified polymorphic markers is amplified by well known amplification techniques, e.g., PCR. Typically, this involves the use of primer sequences that are complementary to the two strands of the target sequence both upstream and downstream from the polymorphism. Asymmetric PCR techniques may also be used. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Although primarily described in terms of a single detection block, e.g., for detection of a single polymorphism, arrays can include multiple detection blocks, and thus be capable of analyzing multiple, specific polymorphisms. In alternate arrangements, it will generally be understood that detection blocks may be grouped within a single array or in multiple, separate arrays so that varying, optimal conditions may be used during the hybridization of the target to the array. For example, it may often be desirable to provide for the detection of those polymorphisms that fall within G-C rich stretches of a genomic sequence, separately from those falling in A-T rich segments. This allows for the separate optimization of hybridization conditions for each situation.

Additional descriptions of the use of oligonucleotide arrays for detection of polymorphisms can be found, for example, in U.S. Pat. Nos. 5,858,659 and 5,837,832, the entire teachings of which are incorporated by reference herein.

Other methods of nucleic acid analysis can be used to detect polymorphisms in seryl tRNA synthetase or variants encoded by seryl tRNA synthetase. Representative methods include direct manual sequencing (Church and Gilbert Proc. Natl. Acad. Sci. USA 81:1991-1995, (1988); Sanger et al., Proc. Natl. Acad. Sci. 74: 5463-5467 (1977); and U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield et al., Proc. Natl. Acad. Sci. USA 86: 232-236 (1991)), mobility shift analysis (Orita et al., Proc. Natl. Acad. Sci. USA 86: 2766-2770 (1989)), restriction enzyme analysis (Flavell et al., Cell 15: 25 (1978); and Geever et al., Proc. Natl. Acad. Sci. USA 78: 5081 (1981)); heteroduplex analysis; chemical mismatch cleavage (CMC) (Cotton et al., Proc. Natl. Acad. Sci. USA 85: 4397-4401 (1985)); RNase protection assays (Myers et al., Science 230: 1242 (1985)); use of polypeptides that recognize nucleotide mismatches, such as E. coli mutS protein; and allele-specific PCR.

In another embodiment of the invention, diagnosis of a susceptibility to an angiogenic disease, a vascular disease, a heart disease, or a circulatory disease can also be made by examining the level of a seryl tRNA synthetase nucleic acid, for example, using in situ hybridization techniques known to one skilled in the art, or by examining the level of expression, activity, and/or composition of a seryl tRNA synthetase polypeptide, by a variety of methods, including enzyme linked immunosorbent assays (ELISAs), Western blots, immunoprecipitations, immunohistochemistry, and immunofluorescence. A test sample from an individual is assessed for the presence of an alteration in the level of a seryl tRNA synthetase nucleic acid or in the expression and/or an alteration in composition of the polypeptide encoded by seryl tRNA synthetase, or for the presence of a particular variant encoded by seryl tRNA synthetase. An alteration in expression of a polypeptide encoded by seryl tRNA synthetase can be, for example, an alteration in the quantitative polypeptide expression (i.e., the amount of polypeptide produced); an alteration in the composition of a polypeptide encoded by seryl tRNA synthetase, or an alteration in the qualitative polypeptide expression (e.g., expression of a mutant seryl tRNA synthetase polypeptide or variant thereof). In a preferred embodiment, diagnosis of a susceptibility to an angiogenic disease, a vascular disease, a heart disease, or a circulatory disease is made by detecting a particular variant encoded by seryl tRNA synthetase, or a particular pattern of variants. Altered levels of seryl tRNA synthetase or altered expression or activity of a seryl tRNA synthetase polypeptide, relative to a control sample, for example, a sample known not to be associated with an angiogenic disease, a vascular disease, a heart disease, or a circulatory disease, indicates an altered susceptibility or likelihood that the individual has an angiogenic disease, a vascular disease, a heart disease, or a circulatory disease.

Both quantitative and qualitative alterations can also be present. An “alteration” or “modulation” in the polypeptide expression, activity, or composition, as used herein, refers to an alteration in expression or composition in a test sample, as compared with the expression or composition of a seryl tRNA synthetase polypeptide in a control sample. A control sample is a sample that corresponds to the test sample (e.g., is from the same type of cells), and is from an individual who is not affected by an angiogenic disease, a vascular disease, a heart disease, or a circulatory disease. An alteration in the expression or composition of the polypeptide in the test sample, as compared with the control sample, is indicative of an altered susceptibility to an angiogenic disease, a vascular disease, a heart disease, or a circulatory disease. Similarly, the presence of one or more different variants in the test sample, or the presence of significantly different amounts of different variants in the test sample, as compared with the control sample, is indicative of an altered susceptibility to an angiogenic disease, a vascular disease, a heart disease, or a circulatory disease.

It is understood that alterations or modulations in polypeptide expression or function can occur in varying degrees. For example, an alteration or modulation in expression can be an increase, for example, by at least 1.5-fold to 2-fold, at least 3-fold, or, at least 5-fold, relative to the control. Alternatively, the alteration or modulation in polypeptide expression can be a decrease, for example, by at least 10%, at least 40%, 50%, or 75%, or by at least 90%, relative to the control.

Various means of examining expression or composition of the seryl tRNA synthetase polypeptide can be used, including spectroscopy, colorimetry, electrophoresis, isoelectric focusing, and immunoassays (e.g., David et al., U.S. Pat. No. 4,376,110) such as immunoblotting (see also Ausubel et al., supra; particularly chapter 10). For example, in one embodiment, an antibody capable of binding to the polypeptide (e.g., as described above), preferably an antibody with a detectable label, can be used. Antibodies can be polyclonal, or more preferably, monoclonal. An intact antibody, or a fragment thereof (e.g., Fab or F(ab′)2) can be used. The term “labeled,” with regard to the antibody, is intended to encompass direct labeling of the antibody by coupling (i.e., physically linking) a detectable substance to the antibody, as well as indirect labeling of the antibody by reacting it with another reagent that is directly labeled. An example of indirect labeling is detection of a primary antibody using a fluorescently labeled secondary antibody.

Western blotting analysis, using an antibody as described above that specifically binds to a mutant seryl tRNA synthetase polypeptide, or an antibody that specifically binds to a non-mutant seryl tRNA synthetase polypeptide, or an antibody that specifically binds to a particular variant encoded by seryl tRNA synthetase, can be used to identify the presence in a test sample of a particular variant of a polypeptide encoded by a polymorphic or mutant seryl tRNA synthetase, or the absence in a test sample of a particular variant or of a polypeptide encoded by a non-polymorphic or non-mutant gene. The presence of a polypeptide encoded by a polymorphic or mutant gene, or the absence of a polypeptide encoded by a non-polymorphic or non-mutant gene, is diagnostic of an altered susceptibility to an angiogenic disease, a vascular disease, a heart disease, or a circulatory disease, as is the presence (or absence) of particular variants encoded by the seryl tRNA synthetase nucleic acid molecule.

In one embodiment of this method, the level or amount of seryl tRNA synthetase polypeptide in a test sample is compared with the level or amount of the seryl tRNA synthetase polypeptide in a control sample. A level or amount of the polypeptide in the test sample that is higher or lower than the level or amount of the polypeptide in the control sample, such that the difference is statistically significant, is indicative of an alteration in the expression of the seryl tRNA synthetase polypeptide, and is diagnostic for an altered susceptibility to an angiogenic disease, a vascular disease, a heart disease, or a circulatory disease.

Alternatively, the composition of the seryl tRNA synthetase polypeptide in a test sample is compared with the composition of the seryl tRNA synthetase polypeptide in a control sample. A difference in the composition of the polypeptide in the test sample, as compared with the composition of the polypeptide in the control sample (e.g., the presence of different variants), is diagnostic for an altered susceptibility to an angiogenic disease, a vascular disease, a heart disease, or a circulatory disease. In another embodiment, both the level or amount and the composition of the polypeptide can be assessed in the test sample and in the control sample. A difference in the amount or level of the polypeptide in the test sample, compared to the control sample; a difference in composition in the test sample, compared to the control sample; or both a difference in the amount or level, and a difference in the composition, is indicative of an altered susceptibility to an angiogenic disease, a vascular disease, a heart disease, or a circulatory disease.

Kits (e.g., reagent kits) useful in the methods of diagnosis comprise components useful in any of the methods described herein, including, for example, hybridization probes or primers as described herein (e.g., labeled probes or primers), reagents for detection of labeled molecules, restriction enzymes (e.g., for RFLP analysis), allele-specific oligonucleotides, antibodies that bind to a mutant or to non-mutant (native) seryl tRNA synthetase polypeptide, means for amplification of nucleic acids comprising seryl tRNA synthetase, or means for analyzing the nucleic acid sequence of seryl tRNA synthetase, or for analyzing the amino acid sequence of a seryl tRNA synthetase polypeptide, etc.

Screening Assays and Agents Identified Thereby

The invention provides methods (also referred to herein as “screening assays”) for identifying the presence of a nucleic acid of the invention, as well as for identifying the presence of a polypeptide encoded by a nucleic acid of the invention. In one embodiment, the presence (or absence) of a nucleic acid molecule of interest (e.g., a nucleic acid that has significant homology with a nucleic acid of seryl tRNA synthetase) in a sample can be assessed by contacting the sample with a nucleic acid comprising a nucleic acid of the invention (e.g., a nucleic acid having the sequence of SEQ ID NO: 2, which may optionally comprise at least one polymorphism, or the complement thereof, or a nucleic acid encoding an amino acid having the sequence of SEQ ID NO: 1, or a fragment or variant of such nucleic acids), under stringent conditions as described above, and then assessing the sample for the presence (or absence) of hybridization. In a preferred embodiment, high stringency conditions are conditions appropriate for selective hybridization. In another embodiment, a sample containing the nucleic acid molecule of interest is contacted with a nucleic acid containing a contiguous nucleotide sequence (e.g., a primer or a probe as described above) that is at least partially complementary to a part of the nucleic acid molecule of interest (e.g., a seryl tRNA synthetase nucleic acid), and the contacted sample is assessed for the presence or absence of hybridization. In a preferred embodiment, the nucleic acid containing a contiguous nucleotide sequence is completely complementary to a part of the nucleic acid molecule of seryl tRNA synthetase.

In any of the above embodiments, all or a portion of the nucleic acid of interest can be subjected to amplification prior to performing the hybridization.

In another embodiment, the presence (or absence) of a seryl tRNA synthetase polypeptide, such as a polypeptide of the invention or a fragment or variant thereof, in a sample can be assessed by contacting the sample with an antibody that specifically binds to the polypeptide of seryl tRNA synthetase (e.g., an antibody such as those described above), and then assessing the sample for the presence (or absence) of binding of the antibody to the seryl tRNA synthetase polypeptide.

In another embodiment, the invention provides methods for identifying agents or compounds (e.g., fusion proteins, polypeptides, peptidomimetics, prodrugs, receptors, binding agents, antibodies, small molecules or other drugs, or ribozymes) that alter or modulate (e.g., increase or decrease) the activity of the polypeptides described herein, or that otherwise interact with the polypeptides herein. For example, such compounds can be compounds or agents that bind to polypeptides described herein (e.g., seryl tRNA synthetase substrates or binding agents); that have a stimulatory or inhibitory effect on, for example, the activity of the polypeptides of the invention; or that change (e.g., enhance or inhibit) the ability of the polypeptides of the invention to interact with molecules with which seryl tRNA synthetase polypeptides normally interact (seryl tRNA synthetase binding agents); or that alter post-translational processing of the seryl tRNA synthetase polypeptide (e.g., agents that alter proteolytic processing to direct the polypeptide from where it is normally synthesized to another location in the cell, such as the cell surface; or agents that alter proteolytic processing such that more polypeptide is released from the cell, etc.).

The candidate compound can cause an increase in the activity of the polypeptide. For example, the activity of the polypeptide can be increased by at least 1.5-fold to 2-fold, at least 3-fold, or, at least 5-fold, relative to the control. Alternatively, the polypeptide activity can be a decrease, for example, by at least 10%, at least 20%, 40%, 50%, or 75%, or by at least 90%, relative to the control.

In one embodiment, the invention provides assays for screening candidate compounds or test agents to identify compounds that bind to or modulate the activity of polypeptides described herein (or biologically active portion(s) thereof), as well as agents identifiable by the assays. As used herein, a “candidate compound” or “test agent” is a chemical molecule, be it naturally-occurring or artificially-derived, and includes, for example, peptides, proteins, synthesized molecules, for example, synthetic organic molecules, naturally-occurring molecule, for example, naturally occurring organic molecules, nucleic acid molecules, and components thereof.

In general, candidate compounds for uses in the present invention may be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are generated, if desired, according to methods known in the art, e.g., by standard extraction and fractionation methods. For example, candidate compounds can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to polypeptide libraries, while the other four approaches are applicable to polypeptide, non-peptide oligomer or small molecule libraries of compounds (Lam, Anticancer Drug Des. 12: 145 (1997)). Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activities should be employed whenever possible.

When a crude extract is found to modulate (i.e., stimulate or inhibit) the expression and/or activity of the nucleic acids and or polypeptides of the present invention, further fractionation of the positive lead extract is necessary to isolate chemical constituents responsible for the observed effect. Thus, the goal of the extraction, fractionation, and purification process is the careful characterization and identification of a chemical entity within the crude extract having an activity that stimulates or inhibits nucleic acid expression, polypeptide expression, or polypeptide biological activity. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases in which it is desirable to alter the activity or expression of the nucleic acids or polypeptides of the present invention.

In one embodiment, to identify candidate compounds that alter the biological activity, for example, the tRNA synthetase enzymatic activity or angiogenic modulatory activity of a seryl tRNA synthetase polypeptide, a cell, tissue, cell lysate, tissue lysate, or solution containing or expressing a seryl tRNA synthetase polypeptide (e.g., SEQ ID NO: 1, or another variant encoded by seryl tRNA synthetase, or a fragment or derivative thereof (as described above), can be contacted with a candidate compound to be tested under conditions suitable for enzymatic reaction or angiogenesis. Methods for assessing seryl tRNA synthetase activity are described, for example, by Sampson and Saks, Nucleic Acids Res. 21(19):4467-75 (1993); and Stefanska et al., J. Antibiot. (Tokyo) 53(12): 1346-53 (2000), the entire teachings of which are incorporated by reference herein. In vitro and in vivo methods for detecting and/or measuring angiogenic activity are described, for example by McCarty et al., Int. J. Oncol. 21(l):5-10 (2002); Blacher et al., Angiogenesis 4(2):133-42 (2001); and Brown Lab Invest. 75(4):539-55 (1996).

Alternatively, the polypeptide can be contacted directly with the candidate compound to be tested. The level (amount) of seryl tRNA synthetase biological activity is assessed (e.g., the level (amount) of seryl tRNA synthetase biological activity is measured, either directly or indirectly), and is compared with the level of biological activity in a control (i.e., the level of activity of the seryl tRNA synthetase polypeptide or active fragment or derivative thereof in the absence of the candidate compound to be tested, or in the presence of the candidate compound vehicle only). If the level of the biological activity in the presence of the candidate compound differs, by an amount that is statistically significant, from the level of the biological activity in the absence of the candidate compound, or in the presence of the candidate compound vehicle only, then the candidate compound is a compound that alters the biological activity of a seryl tRNA synthetase polypeptide. For example, an increase in the level of seryl tRNA synthetase enzymatic activity or angiogenic activity relative to a control, indicates that the candidate compound is a compound that enhances (is an agonist of) seryl tRNA synthetase activity. Similarly, a decrease in the enzymatic activity or angiogenic activity of seryl tRNA synthetase activity relative to a control, indicates that the candidate compound is a compound that inhibits (is an antagonist of) seryl tRNA synthetase activity. In another embodiment, the level of biological activity of a seryl tRNA synthetase polypeptide or derivative or fragment thereof in the presence of the candidate compound to be tested, is compared with a control level that has previously been established. A level of the biological activity in the presence of the candidate compound that differs from the control level by an amount that is statistically significant indicates that the compound alters seryl tRNA synthetase biological activity.

The present invention also relates to an assay for identifying compounds that alter the expression of a seryl tRNA synthetase nucleic acid molecule (e.g., antisense nucleic acids, fusion proteins, polypeptides, peptidomimetics, prodrugs, receptors, binding agents, antibodies, small molecules or other drugs, or ribozymes) that alter (e.g., increase or decrease) expression (e.g., transcription or translation) of the nucleic acid molecule or that otherwise interact with the nucleic acids described herein, as well as compounds identifiable by the assays. For example, a solution containing a nucleic acid encoding a seryl tRNA synthetase polypeptide can be contacted with a candidate compound to be tested. The solution can comprise, for example, cells containing the nucleic acid or cell lysate containing the nucleic acid; alternatively, the solution can be another solution that comprises elements necessary for transcription/translation of the nucleic acid. Cells not suspended in solution can also be employed, if desired. The level and/or pattern of seryl tRNA synthetase expression (e.g., the level and/or pattern of mRNA or of protein expressed, such as the level and/or pattern of different variants) is assessed, and is compared with the level and/or pattern of expression in a control (i.e., the level and/or pattern of seryl tRNA synthetase expression in the absence of the candidate compound, or in the presence of the candidate compound vehicle only). If the level and/or pattern in the presence of the candidate compound differs, by an amount or in a manner that is statistically significant, from the level and/or pattern in the absence of the candidate compound, or in the presence of the candidate compound vehicle only, then the candidate compound is a compound that alters the expression of seryl tRNA synthetase. Enhancement of seryl tRNA synthetase expression indicates that the candidate compound is an agonist of seryl tRNA synthetase activity. Similarly, inhibition of seryl tRNA synthetase expression indicates that the candidate compound is an antagonist of seryl tRNA synthetase activity. In another embodiment, the level and/or pattern of a seryl tRNA synthetase polypeptide(s) (e.g., different variants) in the presence of the candidate compound to be tested, is compared with a control level and/or pattern that has previously been established. A level and/or pattern in the presence of the candidate compound that differs from the control level and/or pattern by an amount or in a manner that is statistically significant indicates that the candidate compound alters seryl tRNA synthetase expression.

In another embodiment of the invention, compounds that alter the expression of a seryl tRNA synthetase nucleic acid molecule or that otherwise interact with the nucleic acids described herein, can be identified using a cell, cell lysate, or solution containing a nucleic acid encoding the promoter region of the seryl tRNA synthetase gene operably linked to a reporter gene. After contact with a candidate compound to be tested, the level of expression of the reporter gene (e.g., the level of mRNA or of protein expressed) is assessed, and is compared with the level of expression in a control (i.e., the level of the expression of the reporter gene in the absence of the candidate compound, or in the presence of the candidate compound vehicle only). If the level in the presence of the candidate compound differs, by an amount or in a manner that is statistically significant, from the level in the absence of the candidate compound, or in the presence of the candidate compound vehicle only, then the candidate compound is a compound that alters the expression of seryl tRNA synthetase, as indicated by its ability to alter expression of a gene that is operably linked to the seryl tRNA synthetase gene promoter. Enhancement of the expression of the reporter indicates that the compound is an agonist of seryl tRNA synthetase activity. Similarly, inhibition of the expression of the reporter indicates that the compound is an antagonist of seryl tRNA synthetase activity. In another embodiment, the level of expression of the reporter in the presence of the candidate compound to be tested, is compared with a control level that has previously been established. A level in the presence of the candidate compound that differs from the control level by an amount or in a manner that is statistically significant indicates that the candidate compound alters seryl tRNA synthetase expression.

Compounds that alter the amounts of different variants encoded by seryl tRNA synthetase (e.g., a compound that enhances activity of a first variant, and that inhibits activity of a second variant), as well as compounds that are agonists of activity of a first variant and antagonists of activity of a second variant, can easily be identified using these methods described above.

In one example, a cell or tissue that expresses or contains a compound that interacts with seryl tRNA synthetase (herein referred to as a “seryl tRNA synthetase substrate,” which can be a polypeptide or other molecule that interacts with seryl tRNA synthetase) is contacted with seryl tRNA synthetase in the presence of a candidate compound, and the ability of the candidate compound to alter the interaction between seryl tRNA synthetase and the seryl tRNA synthetase substrate is determined, for example, by assaying activity of the polypeptide. Alternatively, a cell lysate or a solution containing the seryl tRNA synthetase substrate, can be used. A compound that binds to seryl tRNA synthetase or the seryl tRNA synthetase substrate can alter the interaction by interfering with, or enhancing the ability of seryl tRNA synthetase to bind to, associate with, or otherwise interact with the seryl tRNA synthetase substrate.

Determining the ability of the candidate compound to bind to seryl tRNA synthetase or a seryl tRNA synthetase substrate can be accomplished, for example, by coupling the candidate compound with a radioisotope or enzymatic label such that binding of the candidate compound to the polypeptide can be determined by detecting the label, for example, ¹²⁵I, ³⁵S, ¹⁴C, or ³H, either directly or indirectly, and the radioisotope detected by direct counting of radioemmission or by scintillation counting. Alternatively, candidate compound can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a candidate compound to interact with the polypeptide without the labeling of any of the interactants. For example, a microphysiometer can be used to detect the interaction of a candidate compound with seryl tRNA synthetase or a seryl tRNA synthetase substrate without the labeling of either the candidate compound, seryl tRNA synthetase, or the seryl tRNA synthetase substrate (McConnell et al., Science 257: 1906-1912 (1992)). As used herein, a “microphysiometer” (e.g., CYTOSENSOR™) is an analytical instrument that measures the rate at which a cell acidifies its environment using a light-addressable potentiometric sensor (LAPS). Changes in this acidification rate can be used as an indicator of the interaction between ligand and polypeptide.

In another embodiment of the invention, assays can be used to identify polypeptides that interact with one or more seryl tRNA synthetase polypeptides, as described herein. For example, a yeast two-hybrid system such as that described by Fields and Song (Fields and Song, Nature 340: 245-246 (1989)) can be used to identify polypeptides that interact with one or more seryl tRNA synthetase polypeptides. In such a yeast two-hybrid system, vectors are constructed based on the flexibility of a transcription factor that has two functional domains (a DNA binding domain and a transcription activation domain). If the two domains are separated but fused to two different proteins that interact with one another, transcriptional activation can be achieved, and transcription of specific markers (e.g., nutritional markers such as His and Ade, or color markers such as lacZ) can be used to identify the presence of interaction and transcriptional activation. For example, in the methods of the invention, a first vector is used that includes a nucleic acid encoding a DNA binding domain and a seryl tRNA synthetase polypeptide, variant, or fragment or derivative thereof, and a second vector is used that includes a nucleic acid encoding a transcription activation domain and a nucleic acid encoding a polypeptide that potentially may interact with the seryl tRNA synthetase polypeptide, variant, or fragment or derivative thereof (e.g., a seryl tRNA synthetase polypeptide substrate or receptor). Incubation of yeast containing the first vector and the second vector under appropriate conditions (e.g., mating conditions such as used in the MATCHMAKER™ system from Clontech) allows identification of colonies that express the markers of seryl tRNA synthetase. These colonies can be examined to identify the polypeptide(s) that interact with the seryl tRNA synthetase polypeptide or fragment or derivative thereof. Such polypeptides may be useful as compounds that alter the activity or expression of a seryl tRNA synthetase polypeptide, as described above.

In more than one embodiment of the above assay methods of the present invention, it may be desirable to immobilize a seryl tRNA synthetase polypeptide, or a seryl tRNA synthetase substrate, or other components of the assay on a solid support, in order to facilitate separation of complexed from uncomplexed forms of one or both of the polypeptides, as well as to accommodate automation of the assay. Binding of a candidate compound to the polypeptide, or interaction of the polypeptide with a substrate in the presence and absence of a candidate compound, can be accomplished in any vessel suitable for containing the reactants. Examples of such vessels include microtitre plates, test tubes, and micro-centrifuge tubes. In one embodiment, a fusion protein (e.g., a glutathione-S-transferase fusion protein) can be provided that adds a domain that allows seryl tRNA synthetase or a seryl tRNA synthetase substrate to be bound to a matrix or other solid support.

In another embodiment, modulators of expression of nucleic acid molecules of the invention are identified in a method wherein a cell, cell lysate, tissue, tissue lysate, or solution containing a nucleic acid encoding seryl tRNA synthetase is contacted with a candidate compound and the expression of appropriate mRNA or polypeptide (e.g., variant(s)) in the cell, cell lysate, tissue, or tissue lysate, or solution, is determined. The level of expression of appropriate mRNA or polypeptide(s) in the presence of the candidate compound is compared to the level of expression of mRNA or polypeptide(s) in the absence of the candidate compound, or in the presence of the candidate compound vehicle only. The candidate compound can then be identified as a modulator of expression based on this comparison. For example, when expression of mRNA or polypeptide is greater (statistically significantly greater) in the presence of the candidate compound than in its absence, the candidate compound is identified as a stimulator or enhancer of the mRNA or polypeptide expression. Alternatively, when expression of the mRNA or polypeptide is less (statistically significantly less) in the presence of the candidate compound than in its absence, the candidate compound is identified as an inhibitor of the mRNA or polypeptide expression. The level of mRNA or polypeptide expression in the cells can be determined by methods described herein for detecting mRNA or polypeptide.

In another embodiment, the invention features a method of identifying a candidate compound that alters the expression level or biological activity of a seryl 5 tRNA synthetase in a zebrafish. The method comprises contacting a zebrafish with a candidate compound. The level of seryl tRNA synthetase mRNA or protein expressed or the biological activity of the protein is assessed, and is compared with the level of expression or biological activity in a control (i.e., the level of the expression or biological activity in the absence of the candidate compound, or in the presence of the candidate compound vehicle only) using, for example, methods described herein. If the level of expression or activity in the presence of the candidate compound differs, by an amount or in a manner that is statistically significant, from the level in the absence of the candidate compound, or in the presence of the candidate compound vehicle only, then the candidate compound is a compound that alters the expression or biological activity of seryl tRNA synthetase. In one embodiment, the biological activity is assessed by detecting an increase or a decrease in circulation of blood throughout the zebrafish body, using for example visual inspection under a microscope. In another embodiment the effect of the candidate compound is determined using confocal microangiography to determine alteration in the zebrafish vasculature (Isogai et al., Dev. Biol. 230(2):278-301 (2001)). In another embodiment, the test zebrafish (administered the candidate compound) is a zebrafish having a mutation in a seryl tRNA synthetase gene, and the controls is a wild-type zebrafish with an unmutated seryl tRNA synthetase gene. In other embodiment, seryl tRNA synthetase expression or biological activity is detected as described herein.

This invention further pertains to novel compounds identified by the above-described screening assays. Accordingly, it is within the scope of this invention to further use a compound identified as described herein in an appropriate animal model. For example, a compound identified as described herein (e.g., a candidate compound that is a modulating compound such as an antisense nucleic acid molecule, a specific antibody, or a polypeptide substrate) can be used in an animal model to determine the efficacy, toxicity, or side effects of treatment with such a compound. Alternatively, a compound identified as described herein can be used in an animal model to determine the mechanism of action of such a compound. Furthermore, this invention pertains to uses of novel compounds identified by the above-described screening assays for treatments as described herein. In addition, a compound identified as described herein can be used to alter activity of a seryl tRNA synthetase polypeptide, or to alter expression of seryl tRNA synthetase, by contacting the polypeptide or the nucleic acid molecule (or contacting a cell comprising the polypeptide or the nucleic acid molecule) with the compound identified as described herein.

Pharmaceutical Compositions

The present invention also pertains to pharmaceutical compositions comprising nucleic acids described herein, particularly nucleotides encoding the polypeptides described herein; comprising polypeptides described herein (e.g., SEQ ID NO: 1, and/or other variants encoded by seryl tRNA synthetase); and/or comprising a compound that alters (e.g., increases or decreases) seryl tRNA synthetase expression or seryl tRNA synthetase polypeptide activity as described herein. For instance, a polypeptide, protein, fragment, fusion protein or prodrug thereof, or a nucleotide or nucleic acid construct (vector) comprising a nucleotide of the present invention, a compound that alters seryl tRNA synthetase polypeptide activity, a compound that alters seryl tRNA synthetase nucleic acid expression, or a seryl tRNA synthetase substrate or binding partner, can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like that do not deleteriously react with the active compounds.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other compounds.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active compound. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., that are, if desired, sterilized or mixed with auxiliary agents, e.g., preservatives, stabilizers, wetting agents, buffers or salts for influencing osmotic pressure, etc. The compound may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Compounds described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The compounds are administered in a therapeutically effective amount. The amount of compounds that will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms of an angiogenic disease, a vascular disease, a heart disease, or a circulatory disease, and should be decided according to the judgment of a practitioner and each patient's circumstances. Effective doses may be extrapolated from dose-response curves derived from in vitro or animal model test systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, that notice reflects approval by the agency of manufacture, use of sale for human administration. The pack or kit can be labeled with information regarding mode of administration, sequence of drug administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the compounds can be separated, mixed together in any combination, present in a single vial or tablet. Compounds assembled in a blister pack or other dispensing means is preferred. For the purpose of this invention, unit dosage is intended to mean a dosage that is dependent on the individual pharmacodynamics of each compound and administered in FDA approved dosages in standard time courses.

Methods of Therapy

The present invention also pertains to methods of treatment (prophylactic, diagnostic, and/or therapeutic) for an angiogenic disease, a vascular disease, a heart disease, or a circulatory disease, using a seryl tRNA synthetase therapeutic compound. An “seryl tRNA synthetase therapeutic compound” is a compound that alters (e.g., enhances or inhibits) seryl tRNA synthetase polypeptide activity and/or seryl tRNA synthetase nucleic acid molecule expression, as described herein (e.g., a seryl tRNA synthetase agonist or antagonist). Seryl tRNA synthetase therapeutic compounds can alter seryl tRNA synthetase polypeptide activity or nucleic acid molecule expression by a variety of means, such as, for example, by providing additional seryl tRNA synthetase polypeptide or by upregulating the transcription or translation of the seryl tRNA synthetase nucleic acid molecule; by altering post-translational processing of the seryl tRNA synthetase polypeptide; by altering transcription of seryl tRNA synthetase variants; or by interfering with seryl tRNA synthetase polypeptide activity (e.g., by binding to a seryl tRNA synthetase polypeptide), or by downregulating the transcription or translation of the seryl tRNA synthetase nucleic acid molecule. Representative seryl tRNA synthetase therapeutic compounds include the following: nucleic acids or fragments or derivatives thereof described herein, particularly nucleotides encoding the polypeptides described herein and vectors comprising such nucleic acids (e.g., a nucleic acid molecule, cDNA, and/or RNA, such as a nucleic acid encoding a seryl tRNA synthetase polypeptide or active fragment or derivative thereof, or an oligonucleotide; for example, SEQ ID NO: 2, which may optionally comprise at least one polymorphism, or a nucleic acid encoding SEQ ID NO: 1, or fragments or derivatives thereof); polypeptides described herein; seryl tRNA synthetase substrates; peptidomimetics; fusion proteins or prodrugs thereof; antibodies (e.g., an antibody to a mutant seryl tRNA synthetase, or an antibody to a non-mutant seryl tRNA synthetase polypeptide, or an antibody to a particular variant encoded by seryl tRNA synthetase, as described above); ribozymes; other small molecules; and other compounds that alter (e.g., enhance or inhibit) seryl tRNA synthetase nucleic acid expression or polypeptide activity, for example, those compounds identified in the screening methods described herein, or that regulate transcription of seryl tRNA synthetase variants (e.g., compounds that affect which variants are expressed, or that affect the amount of each variant that is expressed. More than one seryl tRNA synthetase therapeutic compound can be used concurrently, if desired.

The seryl tRNA synthetase therapeutic compound that is a nucleic acid is used in the treatment of an angiogenic disease, a vascular disease, a heart disease, or a circulatory disease. The term, “treatment” as used herein, refers not only to ameliorating symptoms associated with the disease, but also preventing or delaying the onset of the disease, and also lessening the severity or frequency of symptoms of the disease. The therapy is designed to alter (e.g., inhibit or enhance), replace or supplement activity of a seryl tRNA synthetase polypeptide in an individual. For example, a seryl tRNA synthetase therapeutic compound can be administered in order to upregulate or increase the expression or availability of the seryl tRNA synthetase nucleic acid molecule or of specific variants of seryl tRNA synthetase, or, conversely, to downregulate or decrease the expression or availability of the seryl tRNA synthetase nucleic acid molecule or specific variants of seryl tRNA synthetase. Upregulation or increasing expression or availability of a native seryl tRNA synthetase nucleic acid molecule or of a particular variant could interfere with or compensate for the expression or activity of a defective gene or another variant; downregulation or decreasing expression or availability of a native seryl tRNA synthetase nucleic acid molecule or of a particular variant could minimize the expression or activity of a defective gene or the particular variant and thereby minimize the impact of the defective gene or the particular variant.

The seryl tRNA synthetase therapeutic compound(s) are administered in a therapeutically effective amount (i.e., an amount that is sufficient to treat the disease, such as by ameliorating symptoms associated with the disease, preventing or delaying the onset of the disease, and/or also lessening the severity or frequency of symptoms of the disease). The amount that will be therapeutically effective in the treatment of a particular individual's disorder or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

In one embodiment, a nucleic acid of the invention (e.g., a nucleic acid encoding a seryl tRNA synthetase polypeptide, such as SEQ ID NO: 2, which may optionally comprise at least one polymorphism, or a nucleic acid that encodes a seryl tRNA synthetase polypeptide or a variant, derivative or fragment thereof, such as a nucleic acid encoding the protein of SEQ ID NO: 1) can be used, either alone or in a pharmaceutical composition as described above. For example, seryl tRNA synthetase or a cDNA encoding a seryl tRNA synthetase polypeptide, either by itself or included within a vector, can be introduced into cells (either in vitro or in vivo) such that the cells produce native seryl tRNA synthetase polypeptide. If desired, cells that have been transformed with the gene or cDNA or a vector comprising the gene or cDNA can be introduced (or re-introduced) into an individual affected with the disease. Thus, cells that, in nature, lack native seryl tRNA synthetase expression and activity, or have mutant seryl tRNA synthetase expression and activity, or have expression of a disease-associated seryl tRNA synthetase variant, can be engineered to express a seryl tRNA synthetase polypeptide or an active fragment of a seryl tRNA synthetase polypeptide (or a different variant of a seryl tRNA synthetase polypeptide). In a preferred embodiment, nucleic acid encoding the seryl tRNA synthetase polypeptide, or an active fragment or derivative thereof, can be introduced into an expression vector, such as a viral vector, and the vector can be introduced into appropriate cells in an animal. Other gene transfer systems, including viral and nonviral transfer systems, can be used. Alternatively, nonviral gene transfer methods, such as calcium phosphate coprecipitation, mechanical techniques (e.g., microinjection); membrane fusion-mediated transfer via liposomes; or direct DNA uptake, can also be used to introduce the desired nucleic acid molecule into a cell.

Alternatively, in another embodiment of the invention, a nucleic acid of the invention can be used in “antisense” therapy, in which a nucleic acid (e.g., an oligonucleotide) that specifically hybridizes to the RNA and/or genomic DNA of seryl tRNA synthetase is administered or generated in situ. The antisense nucleic acid that specifically hybridizes to the RNA and/or DNA inhibits expression of the seryl tRNA synthetase nucleic acid molecule, e.g., by inhibiting translation and/or transcription. Binding of the antisense nucleic acid can be by conventional base pair complementarity, or, for example, in the case of binding to DNA duplexes, through specific interaction in the major groove of the double helix.

An antisense construct of the present invention can be delivered, for example, as an expression plasmid as described above. When the plasmid is transcribed in the cell, it produces RNA that is complementary to a portion of the mRNA and/or DNA that encodes a seryl tRNA synthetase polypeptide. Alternatively, the antisense construct can be an oligonucleotide probe which is generated ex vivo and introduced into cells; it then inhibits expression by hybridizing with the mRNA and/or genomic DNA of seryl tRNA synthetase. In one embodiment, the oligonucleotide probes are modified oligonucleotides that are resistant to endogenous nucleases, e.g. exonucleases and/or endonucleases, thereby rendering them stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy are also described, for example, by Van der Krol et al., Biotechniques 6: 958-976 (1988); and Stein et al., Cancer Res 48: 2659-2668 (1988). With respect to antisense DNA, oligodeoxyribonucleotides derived from the translation initiation site, e.g. between the −10 and +10 regions of a seryl tRNA synthetase nucleic acid sequence, are preferred.

To perform antisense therapy, oligonucleotides (RNA, cDNA or DNA) are designed that are complementary to mRNA encoding a seryl tRNA synthetase polypeptide. The antisense oligonucleotides bind to seryl tRNA synthetase mRNA transcripts and prevent translation. Absolute complementarity, although preferred, is not required. A sequence “complementary” to a portion of an RNA, as referred to herein, indicates that a sequence has sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid, as described in detail above. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures.

The oligonucleotides used in antisense therapy can be DNA, RNA, or chimeric mixtures or derivatives or modified versions thereof, single-stranded or double-stranded. The oligonucleotides can be modified at the base moiety, sugar moiety, or phosphate backbone, for example, to improve stability of the molecule, hybridization, etc. The oligonucleotides can include other appended groups such as peptides (e.g. for targeting host cell receptors in vivo), or compounds facilitating transport across the cell membrane (see, e.g., Letsinger et al., Proc. Natl. Acad. Sci. USA 86: 6553-6556 (1989); Lemaitre et al., Proc. Natl. Acad Sci. USA 84: 648-652 (1987); PCT International Publication No. W088/09810)) or the blood-brain barrier (see, e.g., PCT International Publication No. W089/10134), or hybridization-triggered cleavage agents (see, e.g., Krol et al., BioTechniques 6: 958-976 (1988)) or intercalating agents. (See, e.g., Zon, Pharm. Res. 5: 539-549 (1988)). To this end, the oligonucleotide may be conjugated to another molecule (e.g., a peptide, hybridization triggered cross-linking agent, transport agent, hybridization-triggered cleavage agent).

The antisense molecules are delivered to cells that express seryl tRNA synthetase in vivo. A number of methods can be used for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically. Alternatively, in a preferred embodiment, a recombinant DNA construct is utilized in which the antisense oligonucleotide is placed under the control of a strong promoter (e.g., pol III or pol II). The use of such a construct to transfect target cells in the patient results in the transcription of sufficient amounts of single stranded RNAs that will form complementary base pairs with the endogenous seryl tRNA synthetase transcripts and thereby prevent translation of the seryl tRNA synthetase mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art and described above. For example, a plasmid, cosmid, YAC, or viral vector can be used to prepare the recombinant DNA construct that can be introduced directly into the tissue site. Alternatively, viral vectors can be used that selectively infect the desired tissue, in which case administration may be accomplished by another route (e.g., systematically).

Endogenous seryl tRNA synthetase expression can also be reduced by inactivating or “knocking out” seryl tRNA synthetase nucleic acid sequences or their promoters using targeted homologous recombination (e.g., see Smithies et al., Nature 317: 230-234 (1985); Thomas and Capecchi, Cell 51: 503-512 (1987); Thompson et al., Cell 5: 313-321 (1989)). For example, a mutant, non-functional seryl tRNA synthetase (or a completely unrelated DNA sequence) flanked by DNA homologous to the endogenous seryl tRNA synthetase (either the coding regions or regulatory regions of seryl tRNA synthetase) can be used, with or without a selectable marker and/or a negative selectable marker, to transfect cells that express seryl tRNA synthetase in vivo. Insertion of the DNA construct, via targeted homologous recombination, results in inactivation of seryl tRNA synthetase. The recombinant DNA constructs can be directly administered or targeted to the required site in vivo using appropriate vectors, as described above. Alternatively, expression of non-mutant seryl tRNA synthetase can be increased using a similar method. Targeted homologous recombination can be used to insert a DNA construct comprising a non-mutant, functional seryl tRNA synthetase (e.g., a gene having SEQ ID NO: 2, which may optionally comprise at least one polymorphism), or a portion thereof, in place of a mutant seryl tRNA synthetase in the cell, as described above. In another embodiment, targeted homologous recombination can be used to insert a DNA construct comprising a nucleic acid that encodes a seryl tRNA synthetase polypeptide variant that differs from that present in the cell.

Alternatively, endogenous seryl tRNA synthetase expression can be reduced by targeting deoxyribonucleotide sequences complementary to the regulatory region of seryl tRNA synthetase (i.e., the seryl tRNA synthetase promoter and/or enhancers) to form triple helical structures that prevent transcription of seryl tRNA synthetase in target cells in the body. (See generally, Helene Anticancer Drug Des., 6(6): 569-84 (1991); Helene et al., Ann, N.Y. Acad. Sci., 660: 27-36 (1992); and Maher, Bioassays 14(12): 807-15 (1992)). Likewise, the antisense constructs described herein, by antagonizing the normal biological activity of the seryl tRNA synthetase protein, can be used in the manipulation of tissue, e.g., tissue differentiation, both in vivo and for ex vivo tissue cultures. Furthermore, the antisense techniques (e.g., microinjection of antisense molecules, or transfection with plasmids whose transcripts are anti-sense with regard to a seryl tRNA synthetase mRNA or gene sequence) can be used to investigate role of seryl tRNA synthetase in developmental events, as well as the normal cellular function of seryl tRNA synthetase in adult tissue. Such techniques can be utilized in cell culture, but can also be used in the creation of transgenic animals.

In yet another embodiment of the invention, other seryl tRNA synthetase therapeutic compounds as described herein can also be used in the treatment or prevention of an angiogenic disease, a vascular disease, a heart disease, or a circulatory disease. The therapeutic compounds can be delivered in a composition, as described above, or by themselves. They can be administered systemically, or can be targeted to a particular tissue. The therapeutic compounds can be produced by a variety of means, including chemical synthesis; recombinant production; in vivo production (e.g., a transgenic animal, such as U.S. Pat. No. 4,873,316 to Meade et al.), for example, and can be isolated using standard means such as those described herein.

A combination of any of the above methods of treatment (e.g., administration of non-mutant seryl tRNA synthetase polypeptide in conjunction with antisense therapy targeting mutant seryl tRNA synthetase mRNA; administration of a first variant encoded by seryl tRNA synthetase in conjunction with antisense therapy targeting a second encoded by seryl tRNA synthetase, can also be used.

In another embodiment, the invention is directed to seryl tRNA synthetase nucleic acid molecules and seryl tRNA synthetase polypeptides for use as a medicament in therapy. For example, the nucleic acid molecules or polypeptides of the present invention can be used in the treatment of an angiogenic disease, a vascular disease, a heart disease, or a circulatory disease. In addition, the seryl tRNA synthetase nucleic acid molecules and seryl tRNA synthetase polypeptides described herein can be used in the manufacture of a medicament for the treatment of an angiogenic disease, a vascular disease, a heart disease, or a circulatory disease.

The invention will be further described by the following non-limiting examples. The teachings of all publications cited herein are incorporated herein by reference in their entirety.

Exemplification

Method of Making Mutant Zebrafish

Pseudotyped retrovirus was made using the cell line GT/186 (Chen et al., 2002, J. Virol 76: 2192-2198; and PCT Publication No.: WO 00/56874; the entire teachings of which are incorporated herein by reference) which contains the viral genome GT2.0 and expresses the Moloney murine leukemia virus proteins gag and pol. This retrovirus contains a gene trap, that is, a nucleic acid sequence that can only be expressed when it has inserted into a gene; zebrafish or zebrafish embryos that express this nucleic acid sequence are zebrafish or embryos that have the proviral insertions in one or more genes in their genomes. The retrovirus was produced in cells as follows. A plasmid encoding the vesicular stomatitis virus glycoprotein (VSV-G) driven by the CMV promoter was transfected into these cells using Lipofectamine (Life Technologies, Rockville, Md.) as follows. Fifteen centimeter plates of cells at 80-90% confluence were transfected with 7.5 micrograms of plasmid and 50 microliters of Lipofectamine reagent. Forty-eight hours later, the media was collected, filtered through a 0.2 micron filter, and concentrated by centrifugation at 21,000 rpm in an SW28 rotor for 90 minutes at 4° C. followed by resuspension of the pellet in PBS (in 0.1% of the original volume).

The pseudotyped retrovirus was injected into the interior (between the cells) of 500 to 2,000 cell stage zebrafish embryos. These injected embryos grew up to be “founders” and were mosaic for many different proviral insertions; most notably they had mosaic germlines, in which an average of 25 different inserts were transmitted to their progeny (F1 fish), each insert inherited by, on average, 15% of the F1 fish. Thirty-six thousand founders were raised to adulthood.

Founders were either crossed to other founders or crossed to non-transgenic fish and quantitative PCR was used to identify the 3 F1 fish with the most inserts in each family as follows. DNA from tail biopsies of 30 six-week-old fish was extracted by incubation at 55° C. in 50 microliters of 50 mM KCl, 10 mM Tris (pH 8.5), 0.01% gelatin, 0.45% NP-40, 0.45% Tween-20, 5mM EDTA and 200 micrograms/ml Proteinase K for at least 2 hours. Proteinase K was inactivated by incubation at 96° C. for 15 minutes.

One microliter of the tail biopsy DNA was used in a PCR assay using a Perkin-Elmer 7700 sequence detector, Perkin-Elmer TaqMan Master Mix and two sets of primers/probes as follows. For determination of the amount of viral sequence, primers SFG-F (CGCTGGAAAGGACCTTACACA (SEQ ID NO: 3)) and SFG-R (TGCGATGCCGTCTACTTTGA (SEQ ID NO: 4)) were used at 74 nM and SFG probe (FAM-CTGCTGACCACCCCCACCGC-TAMRA (SEQ ID NO: 5)) at 200 nM; for internal reference (total DNA) primers RAG-F (ATTGGAGAAGTCTACCAGAAGCCTAA (SEQ ID NO: 6) and RAG-R (CTTAGTTGCTTGTCCAGGGTTGA (SEQ ID NO: 7)) were used at 150 nM and RAG probe (JOE-GCGCAACGGCGGCGCTC-TAMRA (SEQ ID NO:8)) was used at 200 nM. Reactions were incubated at 50° C. for 2 minutes, 95° C. for 10 minutes, and then cycled 30 times at 95° C. for 15 seconds, 60° C. for 1 minute, with 2 fluorescence reads per 60° C. cycle. The Ct (cycle number at which production of a given amplicon passes a specified threshold in the linear range) for the SFG and RAG amplicons were determined, and the deltaCt (Ct(SFG)—Ct(RAG)) was determined for each sample; the higher this number, the greater the number of proviral inserts. The multi-insert F1 fish with the top 3 values per family were kept and pooled. A total of 6,800 F1 families were analyzed in this way.

Multi-insert F1s were crossed to each other or to non-transgenic fish to generate F2 families which had 10-20 different inserts, each insert in half of the fish. Sibling crosses were then conducted to generate F3 families. The F3 progeny were examined under a dissecting microscope at 1, 2, and 5 days post-fertilization for visible phenotypes.

One F2 family, Number 3817, had some crosses in which approximately 25% of the F3 progeny had a defect in that blood was seen to be circulating in a loop around a very limited portion of the head and through the heart, but not through the remainder of the body. A scanned image of this zebrafish mutant is shown in FIG. 3, where a wild-type zebrafish is shown on top and a mutant zebrafish is shown on the bottom.

Identifying the Mutated Gene

Southern blot analysis on DNA samples from adult fish that either did or did not transmit the above-described mutant phenotype and from the individual mutant embryos described above was used to demonstrate that only one proviral insert segregated with this phenotype as follows. DNA was prepared from either tail biopsies of adult fish or entire 5 day old embryos and incubated overnight at 55° C. in 60 microliters of 100 mM NaCl, 50 mM Tris (pH 8.3), 0.4% SDS, 5 mM EDTA, and 200 micrograms/ml Proteinase K. DNA was precipitated with 60 microliters of isopropanol, washed with 100 microliters of 70% ethanol, and resuspended in 10 mM Tris (pH 8), 1 mM EDTA. Twenty-five percent of the DNA from a tail biopsy or all of the DNA from each 5-day-old embryo were cut with BglII, electrophoresed through 0.8% agarose, blotted onto nylon membrane (Hybond N+, Pharmacia, Piscataway, N.J.), and hybridized to a radiolabeled probe containing sequence between the 5′ LTR and the sole BglII site in the virus. Differentially migrating bands represent different insertion sites; only one band was found to fit the following criteria for being linked to the phenotype: the insertion was always in both parents of pairs that transmitted the phenotype (7 pairs), it was never in both parents of pairs that did not transmit the phenotype (9 pairs), and it was present in all mutant embryos analyzed (approximately 80).

Using DNA samples known either to have this insert or to not have it, a Southern blot was performed as described above except that DNA was cut with TaqI and electrophoresed through 1.5% agarose. The result was used to estimate the distance from the 5′ end of the virus to the first TaqI site in the adjacent genomic DNA to be 100bp. Inverse PCR was then used to clone this junction fragment. One microgram of DNA from one of these samples was digested with TaqI in a 10 microliter reaction, and diluted 5-fold. The TaqI was inactivated by incubation at 80° C. for 20 minutes, and 1 microliter of this was put into a 10 microliter ligation reaction with 100 units ligase (New England Biolabs, Beverly, Mass.) and incubated at 14° C. for 20 hours. One microliter was then used in a PCR reaction using Hi Fidelity Expand enzyme (Roche, Indianapolis, Ind.) and the following primers MSL9 (CCATGCCTTGCAAAATGGCGTTACTTAAGC (SEQ ID NO: 9)) and MSL15 (CCGCAACCCTGGGAGACGTCC (SEQ ID NO: 10)), which should amplify 350 bp of viral sequence, as well as the junction fragment. The cycling profile was 7 cycles of 94° C. for 15 seconds; 72° C. for 2 minutes 30 seconds; 32 cycles of 94° C. for 15 seconds 68° C. for 20 seconds; and 72° C. for 2.5 minutes. Amplification products were electrophoresed through 1.5% agarose and a band of 450 bp was gel purified. This DNA was reamplified with nested primers NU3× (TGATCTCGAGCCAAACCTACAGGTGGGGTC (SEQ ID NO: 11)) and IPP5 (GTGGTTCTGGTAGGAGACGA (SEQ ID NO: 12)), and the resulting PCR product was gel purified and sequenced, using standard methods.

To determine the genomic flanking region of the mutated gene, viral sequences were edited from the sequencing results leaving 99 bp of genomic sequence flanking the 5′ side of the virus. This was confirmed by the use of primer 3817c2 (CGTGACATGACACCGTAAGTGTG (SEQ ID NO: 13)) in a PCR reaction with NU3× (which anneals near the 5′ LTR, oriented to amplify away from the virus); this gave a PCR product from DNA from fish bearing the insert but not from DNA from their siblings that did not contain the insert. The 99 bp sequence was used to query the zebrafish whole genome trace depository at Ensembl (Hubbard et al., Nucleic Acids Research 30:38-41, 2002) and was found to be entirely contained within a 746 bp contig of traces that allowed the prediction of 590 bp on the 3′ side of the virus and an additional 57 bp on the 5′ side. A primer was designed complementary to the DNA presumed to be on the 3′ side of the virus, 3817-23 (GTCCAGGCGGACACAGAATGG (SEQ ID NO: 14)), and this was shown to produce a PCR product with primer IPL3 (TGATCTCGAGTTCCTTGGGAGGGTCTCCTC (SEQ ID NO: 15)), which anneals near the 3′ LTR, oriented to amplify away from the virus) when using DNA from fish bearing the insert but not from DNA from their siblings which did not contain the insert. Furthermore, primers 3817c2, 3817-23, and IPL3 were all used together in a PCR reaction which will give different sized products for chromosomes with or without the proviral insertion to genotype the progeny of crosses as homozygous for the insertion, heterozygous, or homozyous non-insertion. This was used to further demonstrate that the insert segregated with the phenotype: analyzing the DNA from individual mutant embryos and their wild type siblings indicated that 110/110 mutants were homozygous for the insertion while 0 of 96 wild-type siblings were homozygous for the insertion.

Sequence Homology of Mutant Zebrafish Gene to Seryl tRNA Synthetases

Using the genomic flanking sequence as a query against the EST database at NCBI (using the standard default parameters provided with the program), 168 bp were found to be similar to several zebrafish ESTs. The same sequence also was found to be homologous to a number of seryl-tRNA synthetase genes when translated in a blastx query, as described herein. Thus, the mutated gene described herein is named a zebrafish tRNA synthetase gene. Primers were designed in both the 5′ and 3′ reading directions from one of the ESTs and used in an RT-PCR reaction to amplify the entire coding region of the cDNA for this gene, which was then sequenced. The cDNA sequence of the zebrafish seryl tRNA synthetase gene is shown in FIG. 2 (SEQ ID NO: 2). The amino seryl tRNA synthetase polypeptide sequence is shown in FIG. 1 (SEQ ID NO: 1).

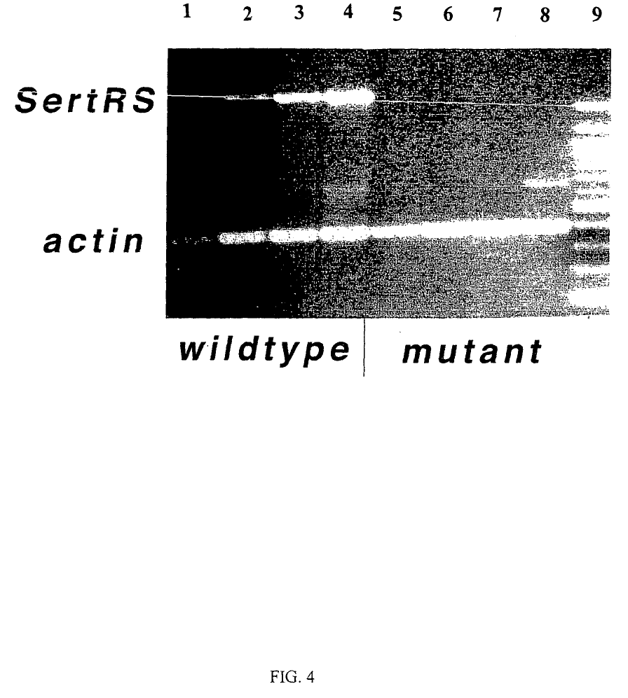

Several sets of primers within the zebrafish seryl tRNA synthetase nucleotide sequence were used to analyze RNA prepared from mutant and wild type embryos by RT-PCR, using standard reaction conditions. FIG. 4 is a scanned image of an agarose gel through which RT-PCR products for seryl tRNA synthetase (SertRS) and actin from 4 day old wild-type and mutant zebrafish have been electrophoresed. Lane 1 shows seryl tRNA synthetase and actin RT-PCR products, diluted 1:1000, from wild-type zebrafish; lane 2 shows seryl tRNA synthetase and actin RT-PCR products, diluted 1:100, from wild-type zebrafish; lane 3 shows seryl tRNA synthetase and actin RT-PCR products, diluted 1:10, from wild-type zebrafish; and lane 4 shows undiluted seryl tRNA synthetase and actin RT-PCR products from wild-type zebrafish. Lane 5 shows seryl tRNA synthetase and actin RT-PCR products, diluted 1:1000, from zebrafish having a mutated seryl tRNA synthetase gene; lane 6 shows seryl tRNA synthetase and actin RT-PCR products, diluted 1:100, from zebrafish having a mutated seryl tRNA synthetase gene; lane 7 shows seryl tRNA synthetase and actin RT-PCR products, diluted 1:10, from zebrafish having a mutated seryl tRNA synthetase gene; and lane 8 shows undiluted seryl tRNA synthetase and actin RT-PCR products from zebrafish having a mutated seryl tRNA synthetase gene. Lane 9 shows a molecular weight ladder. It was found that 4 day old mutant embryos contain less than 1% of the wild type amount of RNA for this gene.

Seryl tRNA Synthetase Function

Seryl tRNA synthetases are involved in a number of biological reactions in the cell. For example, these enzymes activate tRNA molecules by facilitating aminoacylation of tRNA molecules. In addition, aminoacyl tRNA synthetases, including seryl tRNA synthetases produce ApnAs, which are molecules consisting of two adenines joined together by a variable number of phosphates (e.g., 2, 3, 4,5, or 6 phosphates). In the absence of tRNA, the production of ApnAs is increased. In addition, phosphorylation of serine tRNA synthetases enhances production of Ap4A (a molecule consisting of 2 adenines joined together by four phosphates), while have no effect on amino acylation (Dang and Traugh, J. Biol. Chem. 264:5861-5865 (1989)).

ApnAs have been shown to regulate many cellular processes, including vasodilation and vasoconstriction (see, for example, Steinmetz et al., Journal of Pharmacology and Experimental Therapeutics 302: 787-94 (2002); and Steinmetz et al., Journal of Pharmacology and Experimental Therapeutics 294: 1175-81 (2002)). The vasodilation functions appear to be mediated through P2Y purinoreceptors and the vasoconstriction functions seem to be mediated through P2X1 purinoceptors. Thus, it is reasonable to believe that the mutant zebrafish phenotype described herein may be related to altered ApnA activity and/or levels in the fish.

Visual Inspection of Blood Flow in Zebrafish Having a Mutated Seryl tRNA Synthetase Gene

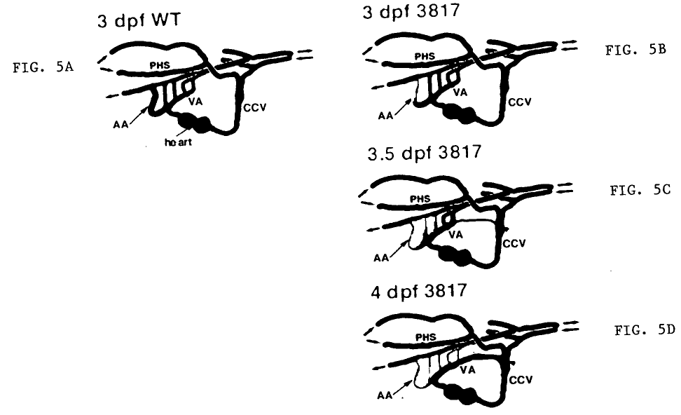

By visual inspection of blood flow under the stereomicroscope, the phenotype for zebrafish having a mutated serine tRNA synthetase gene presents itself fully on day 4 post-fertilization. Using confocal microangiography (as described, for example, by Isogai et al. (Developmental Biology 230(2): 278-301 (2001)), the progression of the phenotype was observed starting at 3 days post-fertilization. Circulation in wild-type zebrafish at 3 days post-fertilization is shown in FIG. 5A. As shown in FIG. 5A, blood flows out of the heart into the ventral aorta (VA) and aortic arch vessels (AA) and then into the head and the trunk of the zebrafish (vessels shown in red). Blood returns through the primary head sinus (PHS) and trunk vessels back into the common cardinal vein (CCV) and into the heart (vessels shown in purple). In zebrafish with a mutated serine tRNA synthetase gene (FIGS. 5B (3 days post-fertilization), 5C (3.5 days post-fertilization), and 5D (4 days post-fertilization), the aortic arch vessels become restricted, preventing blood flow into the head and trunk. In addition, an improper connection is made between the ventral aorta and the common cardinal vein, allowing blood to flow directly from the ventral aorta through the common cardinal vein and into the heart. Starting at 3 days post-fertilization, the restriction of the aortic arch vessels progresses in an anterior to posterior and dorsal to ventral manner, until 4 days post-fertilization, when all of the blood is circulating in a loop formed by the heart, ventral aorta and common cardinal vein. These results demonstrate that zebrafish with a mutated seryl tRNA transferase gene exhibit altered vasculature and altered angiogenic activity.

While this invention has been particularly shown and described with references to preferred embodiments thereof, it will be understood by those skilled in the art that various changes in form and details may be made therein without departing from the scope of the invention.

What is claimed is:

1. An isolated seryl tRNA synthetase polypeptide having at least 82% amino acid identity to the amino acid sequence of SEQ ID NO: 1 or a biologically active fragment thereof.

2. The polypeptide of claim 1, wherein said polypeptide comprises the sequence of SEQ ID NO: 1.

3. The polypeptide of claim 2, wherein said polypeptide consists of the sequence of SEQ ID NO: 1.

4. The polypeptide of claim 1, wherein said polypeptide is a zebrafish polypeptide or a biologically active fragment thereof.

5. An isolated seryl tRNA synthetase polypeptide comprising the sequence of SEQ ID NO: 1 or a biologically active fragment thereof.

6. An isolated seryl tRNA synthetase polypeptide consisting of the sequence of SEQ ID NO: 1 or a biologically active fragment thereof.

7. An isolated polypeptide encoded by the DNA sequence of SEQ ID NO: 2 or a biologically active fragment thereof.

8. An isolated nucleic acid molecule encoding a seryl tRNA synthetase polypeptide, said polypeptide having at least 82% amino acid identity to the amino acid sequence of SEQ ID NO: 1.

9. The isolated nucleic acid molecule of claim 8, wherein said nucleic acid molecule comprises the sequence of SEQ ID NO: 2.

10. The isolated nucleic acid molecule of claim 9, wherein said nucleic acid molecule consists of the sequence of SEQ ID NO: 2.

11. The isolated nucleic acid molecule of claim 8, wherein said nucleic acid molecule is a zebrafish nucleic acid molecule.

12. An isolated nucleic acid molecule encoding the polypeptide of SEQ ID NO: 1, or a biologically active fragment thereof.

13. A vector comprising the nucleic acid molecule of claim 8.

14. A cell comprising the vector of claim 13.

15. A vector comprising the nucleic acid molecule of claim 12.

16. A cell comprising the vector of claim 15.

17. An isolated nucleic acid molecule selected from the group consisting of:
a) the complement of an isolated nucleic acid molecule encoding a seryl tRNA synthetase polypeptide, said polypeptide having at least 82% amino acid identity to the amino acid sequence of SEQ ID NO: 1;
b) the complement of an isolated nucleic acid molecule comprising the sequence of SEQ ID NO: 2;
c) the complement of an isolated nucleic acid consisting of the sequence of SEQ ID NO: 2; and
d) the complement of a nucleic acid molecule encoding the polypeptide of SEQ ID NO: 1.

18. A vector comprising the nucleic acid molecule of claim 17.

19. A cell comprising the vector of claim 18.

20. An isolated nucleic acid molecule selected from the group consisting of:
a) a nucleic acid sequence that is hybridizable under high stringency conditions to a nucleic acid molecule encoding a seryl tRNA synthetase polypeptide, said polypeptide having at least 82% amino acid identity to the amino acid sequence of SEQ ID NO: 1;
b) a nucleic acid sequence that is hybridizable under high stringency conditions to a nucleic acid molecule comprising the sequence of SEQ ID NO: 2;
c) a nucleic acid sequence that is hybridizable under high stringency conditions to a nucleic acid molecule consisting of the sequence of SEQ ID NO: 2; and
d) a nucleic acid molecule that is hybridizable under high stringency conditions to a nucleic acid molecule encoding the polypeptide of SEQ ID NO: 1.

21. A vector comprising the nucleic acid molecule of claim 20.

22. A cell comprising the vector of claim 21.

23. An isolated nucleic acid molecule comprising the sequence of SEQ ID NO: 2.

24. The isolated nucleic acid molecule of claim 23, wherein said nucleic acid molecule consists of the sequence of SEQ ID NO: 2.

25. A vector comprising the nucleic acid molecule of claim 23.

26. A cell comprising the vector of claim 25.

27. A mutated seryl tRNA synthetase gene wherein said mutation results in decreased seryl tRNA synthetase polypeptide activity.

28. The mutated gene of claim 27, wherein said mutation is in an intron of a seryl tRNA synthetase gene.

29. The mutated gene of claim 28, wherein said mutation is a proviral insertion in said intron.

30. A zebrafish comprising a mutated seryl tRNA synthetase gene, wherein the mutation results in decreased seryl tRNA synthetase polypeptide levels in said zebrafish.

31. The zebrafish of claim 30, wherein said mutation results in decreased seryl tRNA synthetase biological activity.

32. The zebrafish of claim 30, wherein said mutation is in an intron of said seryl tRNA synthetase gene.

33. The zebrafish of claim 32, wherein said mutation is a proviral insertion in said intron.

34. The zebrafish of claim 30, said zebrafish comprising a mutation resulting in a phenotype in which blood circulates through the heart of said zebrafish and re-enters the heart, without circulating throughout the trunk of said zebrafish.

35. The zebrafish of claim 34, wherein said phenotype results from altered vasculature.

36. The zebrafish of claim 34, wherein said zebrafish has altered angiogenic activity.

37. An antibody that selectively binds a seryl tRNA synthetase polypeptide, said S polypeptide having at least 82% amino acid identity to SEQ ID NO: 1.

38. A method of identifying a compound that modulates expression of a seryl tRNA synthetase nucleic acid molecule, said nucleic acid molecule encoding a polypeptide having at least 82% amino acid identity to the amino acid sequence of SEQ ID NO: 1, said method comprising:
a) contacting said nucleic acid molecule with a candidate compound under conditions suitable for expression of said nucleic acid molecule; and
b) assessing the level of expression of said nucleic acid molecule, wherein a candidate compound that increases or decreases expression of said seryl tRNA synthetase nucleic acid molecule relative to a control is a compound that modulates expression of said seryl tRNA synthetase nucleic acid molecule.

39. The method of claim 38, wherein said candidate compound is an activator of angiogenic expression.

40. The method of claim 38, wherein said candidate compound is an inhibitor of angiogenic expression.

41. A method of identifying a compound that modulates expression of a seryl tRNA synthetase nucleic acid molecule, said nucleic acid molecule encoding a polypeptide having at least 82% amino acid identity to the amino acid sequence of SEQ ID NO: 1, said method comprising:
a) contacting a cell or animal comprising said nucleic acid molecule with a candidate compound under conditions suitable for expression of said nucleic acid molecule; and
b) assessing the level of expression of said nucleic acid molecule,
wherein a candidate compound that increases or decreases expression of said seryl tRNA synthetase nucleic acid molecule relative to a control is a compound that modulates expression of said seryl tRNA synthetase nucleic acid molecule.

42. The method of claim 41, wherein said animal is a zebrafish.

43. The method of claim 41, wherein said candidate compound is an activator of seryl tRNA synthetase expression.

44. The method of claim 41, wherein said candidate compound is an inhibitor of seryl tRNA synthetase expression.

45. A method of identifying a compound that modulates the biological activity of a seryl tRNA synthetase polypeptide, said polypeptide have at least 82% amino acid identity to the amino acid sequence of SEQ ID NO: 1, said method comprising:
a) contacting said polypeptide or a biologically active fragment thereof with a candidate compound under conditions suitable for seryl tRNA synthetase biological activity; and
b) assessing the seryl tRNA synthetase biological activity of said polypeptide or fragment,
wherein a candidate compound that increases or decreases the seryl tRNA synthetase biological activity level of said polypeptide or biologically active fragment thereof relative to a control is a compound that modulates the seryl tRNA synthetase biological activity of said polypeptide.

46. The method of claim 45, wherein said candidate compound is an activator of seryl tRNA synthetase biological activity.

47. The method of claim 45, wherein said candidate compound is an inhibitor of seryl tRNA synthetase biological activity.

48. A method of identifying a compound that modulates the seryl tRNA synthetase enzymatic activity of a seryl tRNA synthetase polypeptide, said polypeptide have at least 82% amino acid identity to the amino acid sequence of SEQ ID NO: 1, said method comprising:
a) contacting a cell or animal comprising said polypeptide or a biologically active fragment thereof with a candidate compound under conditions suitable for seryl tRNA synthetase enzymatic activity; and
b) assessing the seryl tRNA synthetase enzymatic activity of said polypeptide or fragment,
wherein a candidate compound that increases or decreases the seryl tRNA synthetase enzymatic activity level of said polypeptide or biologically active fragment thereof relative to a control is a compound that modulates the seryl tRNA synthetase activity of said polypeptide.

49. The method of claim 48, wherein said animal is a zebrafish.

50. The method of claim 48, wherein said candidate compound is an inhibitor of seryl tRNA synthetase activity.

51. The method of claim 48, wherein said candidate compound is an activator of seryl tRNA synthetase activity.

52. A method of identifying a compound that modulates the angiogenic activity of a seryl tRNA synthetase polypeptide, said method comprising:
a) contacting said polypeptide or a biologically active fragment thereof with a candidate compound under conditions suitable for angiogenic activity; and
b) assessing the angiogenic activity of said polypeptide or fragment,
wherein a candidate compound that increases or decreases the angiogenic activity level of said polypeptide or biologically active fragment thereof relative to a control is a compound that modulates the angiogenic activity of said polypeptide.

53. The method of claim 52, wherein said candidate compound is an activator of angiogenic activity.

54. The method of claim 52, wherein said candidate compound is an inhibitor of angiogenic activity.

55. A method of identifying a compound that modulates the angiogenic activity of a seryl tRNA synthetase polypeptide, said method comprising:
a) contacting a cell or animal comprising said polypeptide or a biologically active fragment thereof with a candidate compound under conditions suitable for angiogenic activity; and
b) assessing the angiogenic activity of said polypeptide or fragment,
wherein a candidate compound that increases or decreases the angiogenic activity level of said polypeptide or biologically active fragment thereof relative to a control is a compound that modulates the angiogenic activity of said polypeptide.

56. The method of claim 55, wherein said animal is a zebrafish.

57. The method of claim 55, wherein said candidate compound is an inhibitor of angiogenic activity.

58. The method of claim 55, wherein said candidate compound is an activator of angiogenic activity.

59. The method of claim 55, wherein said seryl tRNA synthetase polypeptide is a human seryl tRNA synthetase polypeptide.

60. The method of claim 55, wherein said seryl tRNA synthetase polypeptide has at least 82% amino acid identity to SEQ ID NO: 1.

61. A method of identifying a compound that modulates expression of a nucleic acid molecule encoding a seryl tRNA synthetase polypeptide, said polypeptide having at least 82% amino acid identity to the amino acid sequence of SEQ ID NO: 1, said method comprising:
a) contacting a nucleic acid molecule comprising a promoter region of a nucleic acid molecule encoding a seryl tRNA synthetase polypeptide or functional part of a promoter region of said a nucleic acid molecule encoding a seryl tRNA synthetase polypeptide operably linked to a reporter gene with a candidate compound; and
b) assessing the level of expression of said reporter gene,
wherein a candidate compound that increases or decreases expression of said reporter gene relative to a control is a compound that modulates expression of said nucleic acid molecule encoding a seryl tRNA synthetase polypeptide.

62. The method of claim 61, wherein said method is carried out in a cell.

63. The method of claim 61, wherein said candidate compound is an activator of seryl tRNA synthetase expression.

64. The method of claim 61, wherein said candidate compound is an inhibitor of seryl tRNA synthetase expression.

65. A method of identifying a polypeptide that interacts with a seryl tRNA synthetase polypeptide in a yeast two-hybrid system, said seryl tRNA synthetase polypeptide have at least 82% amino acid identity to the amino acid sequence of SEQ ID NO: 1, said method comprising:
a) contacting a first nucleic acid vector with a second nucleic acid vector in a yeast two-hybrid system, wherein said first nucleic acid vector comprises a nucleic acid molecule encoding a DNA binding domain and a seryl tRNA synthetase polypeptide, and wherein said second nucleic acid vector comprises a nucleic acid encoding a transcription activation domain and a nucleic acid encoding a test polypeptide; and
b) assessing transcriptional activation in said yeast two-hybrid system,
wherein an increase in transcriptional activation relative to a control indicates that the test polypeptide is a polypeptide that interacts with a seryl tRNA synthetase polypeptide.
